(19) United States
(12) Patent Application Publication
(10) Pub. No.: US 2026/0325724 A1
(43) Pub. Date: Oct. 22, 2026

(54) BIOCOMPATIBLE STRUCTURED MATERIAL AND USES THEREOF

(71) Applicants: Universität zu Lübeck, Lübeck (DE); Christian-Albrechts-Universität zu Kiel, Kiel (DE); Phi-Stone AG, Mielkendorf (DE)

(72) Inventors: Anna GAPEEVA, Kiel (DE); Rainer ADELUNG, Kiel (DE); Sören KAPS, Schönkirchen (DE); Jörg BAHR, Altenholz-Klausdorf (DE); Leonard SIEBERT, Kiel (DE); Hartmut SCHMIDT-NIEPENBERG, Ahrensbök (DE); Ala COJOCARU, Kiel (DE); Salvatore GRISANTI, Bad Schwartau (DE); Ayseguel TURA, Lübeck (DE); Judith SEWING, Lübeck (DE); Stefanie GNIESMER, Lübeck (DE); Swaantje GRISANTI, Bad Schwartau (DE); Svenja Rebecca SONNTAG, Lübeck (DE)

(21) Appl. No.: 19/110,500

(22) PCT Filed: Sep. 21, 2023
(86) PCT No.: PCT/EP2023/076043

§ 371 Date: Mar. 11, 2025

(30) Foreign Application Priority Data
Sep. 22, 2022 (EP) 22197123.7

(51) Int. Cl. A61L 31/12; A61F 9/007; A61L 31/06; A61L 31/14

(52) U.S. Cl. CPC A61L 31/128; A61F 9/00781; A61L 31/06; A61L 31/146

(57) ABSTRACT

A biocompatible structured material comprising a polymerized matrix and tetrapodal ZnO microparticles (t-ZnO) and/or fragments thereof, wherein the t-ZnO microparticles have a core and arms having an arm length of about 0.5 μm to about 100 μm, a diameter at the core of about 0.8 μm to about 5 μm, and a diameter at the tip of about 0.05 μm to at most the diameter at the core; the weight fraction of the t-ZnO microparticles and/or fragments thereof is about 20 to about 90 weight percent; and the t-ZnO microparticles and/or fragments thereof are embedded in the polymerized matrix and/or are partially protruding from the matrix on the surface of the biocompatible structured material.

### FIELD OF THE INVENTION

The present invention relates to a biocompatible, antifibrotic structured material for implantable devices, especially for drainage implants in glaucoma surgery. The invention further relates to glaucoma drainage implants comprising said material, and to their preparation and use.

### BACKGROUND OF THE INVENTION

Glaucoma describes a group of disorders associated with the damage of the optic nerve usually caused by an elevated intraocular pressure (IOP). Glaucoma leads to visual field deterioration and eventually to permanent vision loss, as there is currently no treatment to restore the optic nerve damage. Being the second leading cause of blindness after cataracts and the largest cause of irreversible blindness worldwide, glaucoma poses a significant threat to public health and the quality of life. It has been estimated that the total number of glaucoma cases for people aged 40-80 years will increase from 76.0 million in 2020 to 111.8 million in 2040 due to population ageing.

The main risk factor for the development of glaucoma is elevated intraocular pressure, which occurs due to a slowdown in the removal of the eye fluid (aqueous humor). Depending on the appearance of drainage pathways, glaucoma can be divided into open-angle glaucoma (OAG) and angle-closure glaucoma (ACG), with the first type being the most common one. In this case, the angle between the iris and the cornea is open and wide, allowing the aqueous humor to flow its natural path around the lens and iris. However, if the drainage channels (trabecular meshwork and Schlemm's canal) are clogged, the outflow of the aqueous humor is hindered, resulting in raised IOP and subsequent degeneration of the optic nerve. Therefore, most treatments to prevent the progression of glaucoma are focused on continuous IOP reduction. First-line treatments include non-invasive medical therapy (in form of eye drops) and laser treatments, while invasive incisional surgery such as trabeculectomy or implantation of tube shunts is performed in more severe cases or when target IOP level could not be reached by other means. However, these standard techniques suffer from several drawbacks. The effectiveness of topical medications is limited by patient adherence to the medication regimen and by intolerance due to side effects such as ocular surface irritations and allergy. Despite significant IOP reduction up to 35.9%, the effect of laser treatments was found to decrease over time, requiring further medications or surgical intervention after 5 years. Incisional surgeries provide the highest efficiency in lowering IOP to nearly 50% but are associated with a more than 30% postoperative complications rate (e.g., hypotony, bleb leak, or endophthalmitis) and with approximately 20% reoperation rate.

In recent years, minimally invasive glaucoma surgery (MIGS) emerged as an alternative and less invasive surgery to treat OAG. Despite being less effective in reducing IOP than traditional surgery, it is a promising technique for patients with mild to moderate glaucoma and in cases where medical therapy or laser treatments have failed. In contrast to traditional incisional surgeries where conjunctival dissection is required, in MIGS, micro-sized drainage stents can be inserted through a small corneal incision by using an injector, minimizing the trauma to the target tissue, as well as surgical and postoperative recovery time. Depending on the placement and the outflow pathway, microstents used in MIGS can be classified into Schlemm's canal stents, suprachoroidal stents, and subconjunctival stents.

Regardless of the placement, major challenges for MIGS devices are excessive wound healing (fibrotic scarring) and foreign body reaction processes as a response of the vascularized living tissue to injury and implant, causing restriction or complete blockage of the aqueous humor flow and resulting in an increase in IOP. As the success of glaucoma filtering surgeries are limited by postoperative encapsulation, introduction of MIGS has not reduced postoperative fibrosis, and five years after implanting of glaucoma drainage devices the success rate is only 40 to 50%. To reduce postoperative fibrosis, it is essential to influence the natural wound healing mechanisms. In order to prevent excessive scarring, as with traditional incisional surgeries, adjunctive antiproliferative drugs such as mitomycin-C and 5-fluorouracil are used. However, despite the adjunction of these drugs, high rates of postoperative interventions such as bleb needling revisions and reoperations have been reported. As with trabeculectomy, where the use of antifibrotic agents has a long history and is associated with an increased risk of complications, there is a likelihood for the development of thin avascular filtering blebs and endophthalmitis. Furthermore, both antiproliferative substances can cause corneal epithelial toxicity. Therefore, reduction or elimination of antifibrotics is desirable.

Inflammatory wound healing mechanisms caused both by surgery and the introduced materials are major determinant of success or failure in MIGS. Though modern devices consisting of glutaraldehyde cross-linked porcine gelatin (XEN™) or polystyrene-b-isobutylene-b-styrene (Preserflo™) appear to reduce the fibrotic reaction, antifibrotics are still needed also with these devices.

Accordingly, a need exists for materials that can be used for implantable devices to avoid excessive wound healing processes and to overcome one or more of the deficiencies of the prior art, in particular for the treatment of glaucoma in the context of MIGS.

It is an object of the present invention to provide new materials for implantable devices to reduce or eliminate fibrotic and foreign body reaction processes, so as to enable effective, long-term and largely side-effect-free use of such devices. It is another object of the invention to provide a glaucoma drainage implant, particularly for MIGS, which may be implanted into the eye to relieve excess IOP and to treat glaucoma, and that overcomes the disadvantages of conventional glaucoma treatments at least to some extent, further being easy and economical to produce.

### SUMMARY OF THE INVENTION

According to the invention, the objects are achieved by the main features of the claims. For these purposes, the invention provides biocompatible structured materials and implantable devices comprising such biocompatible structured materials.

In accordance with a first aspect of the present invention, there is provided a biocompatible structured material comprising a polymerized matrix and tetrapodal ZnO microparticles (t-ZnO) and/or fragments thereof, which are embedded in the polymerized matrix, and/or which are partially protruding from the matrix on the surface of the biocompatible structured material.

Preferably, the polymerized matrix of the biocompatible structured material comprises an elastomer, such as poly- and/or oligo-siloxanes, and more preferably it comprises polydimethylsiloxane (PDMS).

In one embodiment, there is provided a biocompatible structured material comprising a polymerized matrix having substantially interconnected tunnel-shaped micropores, which is obtainable by partially or totally removing the substantially interconnected network structure of t-ZnO microparticles and/or fragments thereof from the polymerized matrix by acid hydrolysis.

In another aspect, the present invention provides an implantable device comprising the biocompatible structured material comprising a polymerized matrix and tetrapodal t-ZnO microparticles and/or fragments thereof, and/or comprising a polymerized matrix having substantially interconnected tunnel-shaped micropores obtainable by partially or totally removing the substantially interconnected network structure of t-ZnO microparticles and/or fragments thereof from the polymerized matrix.

In one preferred embodiment, the implantable device is a glaucoma drainage implant. Such glaucoma drainage implant may have the shape of a substantially straight, flexible, elongated body with a circular or polygonal cross-section. The body of the glaucoma drainage implant may additionally contain a lumen.

This invention also provides methods for reducing intraocular pressure in an eye of a mammalian subject in need thereof, by implanting one or more implantable devices according to the invention into the eye.

### BRIEF DESCRIPTION OF THE DRAWINGS

FIG. 1 shows scanning electron microscopy images (SEM) of tetrapodal ZnO microparticles (t-ZnO) according to the invention. SEM photomicrographs of t-ZnO at different magnifications: 708× (A), 1440× (B), 350× (C), 1000× (D), and 5310× (E) showing the unique morphological structure of the t-ZnO microparticles. The black line in the graphs has the length of 20 μm (A), 10 μm (B), 50 μm (C), 20 μm (D) and 2 μm (E).

FIG. 2 shows the absorption spectra of t-ZnO suspensions in HTF (human Tenon's fibroblast) culture medium at different concentrations (B-D). A: Statistical analysis shows a significant increase in absorption at 570 nm at t-ZnO concentrations of 100 and 1000 μg/mL compared to untreated culture medium (p<0.05 and <0.001, one way ANOVA, Dunn's Multiple Comparison Test).

FIG. 3 shows MTT test results to determine t-ZnO toxicity in a HTF culture. The half maximal inhibitory concentration (IC50) was 9.4 μg/mL (range: 8.7 to 10.3 μg/mL).

FIG. 4 illustrates the effect of t-ZnO microparticles on HTF proliferation: (A) The control (medium only) shows high Ki67 expression (grey dots represent nuclei of proliferating HTFs). (B-F) Treatment with t-ZnO microparticles results in a reduction of KI67 expression in HTFs, indicating a reduction of cell proliferation. (G) Statistical analysis shows a significant reduction of HTF proliferation with concentrations of 8 and 10 μg/mL t-ZnO microparticles in the medium compared to the control (p<0.05 and <0.001, one way ANOVA, Dunn's multiple comparison Test).

FIG. 5 illustrates the effect of t-ZnO microparticles on HTF contractility (α-SMA expression): (A) HTFs of the control group (medium only) exhibited an intense fibrillar pattern of α-SMA-specific proteins. (B-D) Treatment with t-ZnO microparticles results in a reduction of α-SMA expression in HTFs, indicating a reduction in cell contractility (A: 0 μg/mL, B: 5 μg/mL. C: 10 μg/mL, D: 20 μg/mL). (E) Statistical analysis shows a significant reduction of HTF contractility

at a concentration of 20 μg/mL t-ZnO microparticles in the medium compared to the control (p<0.001, one way ANOVA, Dunn's multiple comparison Test).

FIG. 6 illustrates the effect of t-ZnO microparticles on HTF transdifferentiation (p-SMAD expression): (A) HTFs of the control group (medium only) showed many p-SMAD-positive cells. (B-D) Treatment of HTFs with t-ZnO microparticles results in a reduction of p-SMAD expression, indicating decreased cell transdifferentiation. (A: 0 μg/mL, B: 2 μg/mL, C: 4 μg/mL, D: 6 μg/mL, E: 8 μg/mL, F: 10 μg/mL) (G) Statistical analysis shows a significant reduction of HTF transdifferentiation at concentrations of 8 and 10 μg/mL t-ZnO microparticles compared to the control (p<0.05, p<0.01, One way ANOVA, Dunn's multiple comparison Test).

FIG. 7 illustrates wound healing rates 24 and 48 hours after treatment with t-ZnO at different concentrations: (A) The wound gap areas were calculated following 24- and 48-hours treatment. Wound gaps in untreated controls (0 μg/mL) and 1 μg/mL concentrations are almost closed at 48 hours. Concentrations of 5 μg/mL keep the wound gap open over 48 hours. Concentrations of 10 and 20 μg/mL led to a widened wound gap due to toxic effects. (B) The cell free wound area at 24 and 48 hours was correlated to the initial double wound area to consider lost cells at the wound margins. Lower proliferation rates at 48 hours after treatment with 10 and 20 μg/mL t-ZnO may be explained by toxic effects.

FIG. 8 shows cytokine levels in HTFs 24 hours (A-E) and 48 hours (F-J) after treatment with t-ZnO at different concentrations. PT=cytokine levels in HTFs before the t-ZnO treatment.

FIG. 9A is a schematic illustration of the custom-made extrusion device for drainage implant (stent) preparation. The polymer/t-ZnO mixture was loaded into the extrusion device, conveyed inside the cylinder and pressed out of a nozzle. The pressure was generated with a piston. FIG. 9B shows a photograph of ready to use drainage implants. FIG. 9C shows a drainage implant after tensile test. The drainage implant was fixed on a 3D printed sample holder, which was cut in the middle after being mounted into the tensile test machine and before starting the tensile test. FIG. 9D shows a sample for in vitro testing. Drainage implants of the same material composition/variation were placed next to each other on a glass slide with a thin PDMS coating.

FIG. 10A is a schematic illustration of a drainage implant according to the invention (L_{di}=length of the drainage implant; d_{L}=diameter of the lumen; dₐ=outer diameter of the drainage implant). 1 and 2 represent examples of partially protruding t-ZnO microparticle arms. B is schematic illustration of a t-ZnO microparticle according to the invention (Lₐᵣₘ=arm length; d_{core}=diameter at the core; dₜᵢₚ=a diameter at the tip). C is a SEM micrograph of a drainage implant according to the invention showing a magnified view of the implant's surface, in which t-ZnO microparticles partially protruding from the polymerized matrix are visible.

FIG. 11 presents SEM micrographs of drainage implants containing different amounts of t-ZnO microparticles. Drainage implants produced with a 400 μm nozzle are shown at a magnification of A 500×, and B 3500× (top view), and at a magnification of C 500×, and D 1500× (cross-section). Drainage implants produced with a 200 μm nozzle are shown at a magnification of E 500× (top view), and F 1500× (cross-section). The black line in the graphs has the length of 100 μm (A), 10 μm (B), 100 μm (C), 20 μm (D) and 50 μm (E) and (F).

FIG. 12A is a SEM micrograph of a cross-section of a drainage implant with a lumen. B is a magnified image of the indicated section of A. C and D represent energy-dispersive X-ray spectroscopy (EDX)-analysis results illustrating the determined distribution of the elements in a material cross-section of the implant according to the invention. Here, the presence of zinc (Zn; C) and silicon (Si; D) is shown to be associated with the presence of t-ZnO microparticles and [SiR₂O]ₙ. SEM/EDX-images were obtained with a Zeiss Ultra Plus (Carl Zeiss Microscopy GmbH, Jena, Germany) SEM with an EDX-unit (Oxford Instruments).

FIG. 13 is a graph illustrating the mechanical properties of drainage implants derived from the tensile test. All results are represented by mean values and standard deviations of 5 measurements for each material composition/variation tested.

FIG. 14 is a graph illustrating cell viability of rat embryonic fibroblasts on drainage implants in correlation with roughness (Rz) and released Zn ion concentration. All results are represented by mean values and standard deviations of three samples. The statistical analysis shows a significant reduction in cell viability on implants containing 60 wt % and 75 wt % (p<0.001, indicated by three asterisks) as well as on etched implants (p<0.01, indicated by two asterisks) compared to the control.

FIG. 15 is a photograph of a water droplet (stained with methylene blue) placed on drainage implants containing 75 wt % t-ZnO. The combination of the hydrophobic PDMS matrix and the roughness created by protruding t-ZnO particles makes the surface superhydrophobic so that the water droplet remains nearly spherical.

FIG. 16 depicts the in vivo position of drainage implants according to the invention 2-3 mm into the anterior chamber and at the distal end under the conjunctiva in a rabbit eye.

FIG. 17 shows the intraocular pressure (IOP) R/L ratio in the eyes of rabbits upon insertion of drainage implants according to the invention. IOP was measured as IOP ratio between experimental right (R) and control left (L) eye immediately before (day 0) as well as after surgery on days 1, 3, 7, 10, and 14. A: G1 Implant with 200 μm outer diameter. B: G2 Implant with 400 μm diameter. Statistically significant values are marked with *.

FIG. 18 shows IOP R/L ratio in the eyes of rabbits upon insertion of drainage implants according to the invention. IOP was measured as IOP ratio between experimental right (R) and control left (L) eye immediately before (day 0) as well as after surgery on days 1, 3, 7, 10, 14, 17, 21, 24, 28, 31, 35, 38 and 42. G3 Implant with 200 μm outer diameter. Statistically significant values are marked with *.

FIG. 19 shows IOP R/L ratio in the eyes of rabbits upon insertion of drainage implants according to the invention. IOP was measured as IOP ratio between experimental right (R) and control left (L) eye immediately before (day 0) as well as after surgery on days 1, 3, 7, 10, 14, 17, 21, 24, 28, 31, 35, 38 and 42. G4 Implant with 400 μm outer diameter. Statistically significant values are marked with *.

FIG. 20 depicts eyes of rabbits upon insertion of drainage implants according to the invention. After two (A) and after six (B) weeks, the clinical examination of the inserted implants showed neither toxic changes nor inflammatory reactions in the eyes.

FIG. 21 depicts the histological analysis of the implantation sites. A-D: two weeks and E-H: six weeks after insertion of the drainage implant. Left column=hematoxylin and eosin staining; right column=Masson staining.

### DETAILED DESCRIPTION OF THE INVENTION

ZnO is widely used in pharmaceutical products (e.g., as an antibacterial and UV absorbing component in creams) and is actively investigated for use in various biomedical applications (e.g., cancer treatment, periodontal membrane, anti-biofilm materials, and antivirals). In these contexts, the shape and the size of ZnO particles have been shown to influence their biological effects. It has been demonstrated that the incorporation of low amounts of tetrapodal shaped ZnO (t-ZnO) into 3D membranes supports the growth of eukaryotic cells and promotes osteogenesis, while increased t-ZnO concentrations may lead to cell inhibitory effects. The cytotoxicity of t-ZnO microparticles was found to be lower compared to ZnO nanoparticles due to their larger size (i.e., the inability of cellular uptake) and therefore to lower surface-to-volume ratios (i.e., lower surface reactivity).

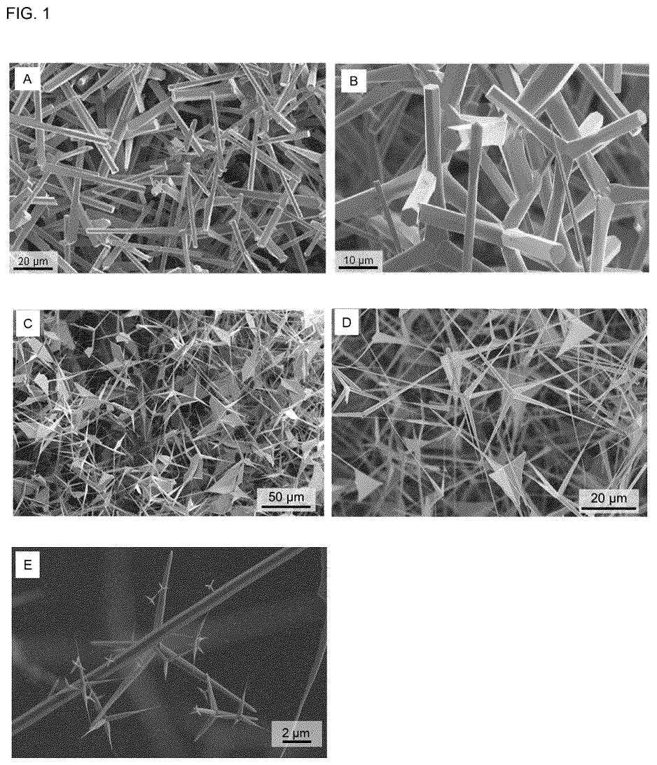

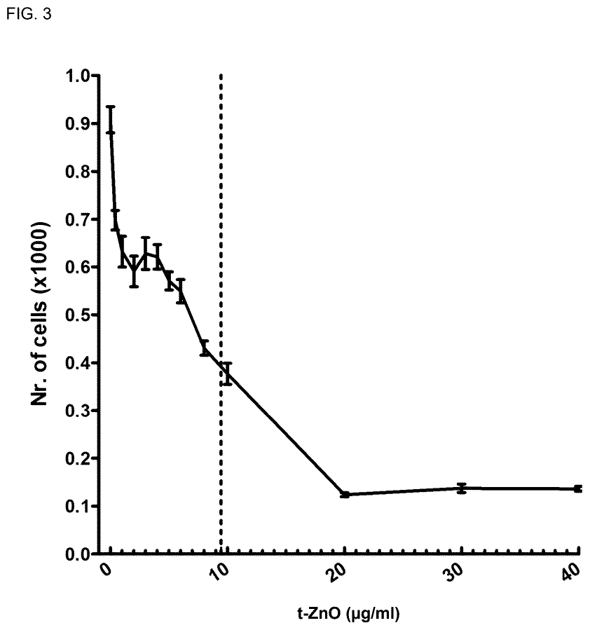

t-ZnO microparticles (as illustrated in FIG. 1 and described in more detail in the Examples section), both in an intact state and as fragments resulting from the breaking of one or more arms of the tetrapodal structure, represent a biocompatible material, which, on the one hand, may inhibit cell proliferation, and, on the other hand, does not cause an unacceptable degree of damage to cells or tissues. The dimensions of the t-ZnO microparticles (which can be varied depending on the synthesis parameters, e.g., temperature and duration, and/or on the growth technique), as well as their concentration and spatial distribution in relation to the cell material/tissue crucially determine the biological properties of the t-ZnO, as exemplified in FIGS. 3 to 8 and presented in the corresponding parts of the Examples section.

Combining t-ZnO microparticles with an inert support allows the production of structured materials with substantially defined amounts and substantially defined spatial distributions of t-ZnO. Such materials make it possible to localize the application of the t-ZnO microparticles and to control their effects in a targeted manner, e.g., in a biological tissue or in an organ. Moreover, the overall structural features of the resulting biocompatible structured material may further influence the behavior of the biological tissue, leading to the desired antifibrotic properties.

Matrices comprising t-ZnO microparticles have already been used, e.g., as an antifouling yarn (WO2021228322A1).

The invention provides a biocompatible structured material comprising a polymerized matrix and t-ZnO microparticles and/or fragments thereof. The material of the invention comprises t-ZnO microparticles having a core and arms having an arm length of about 0.5 μm to about 100 μm, a diameter at the core of about 0.8 μm to about 5 μm, and a diameter at the tip of about 0.05 μm to at most the diameter at the core. At the core of the particles, the arms meet. The tetrapodal ZnO microparticles have a tetrahedron angle between their arms. Fragments of such microparticles typically result from the breaking of one or more arms of the tetrapodal structure. Preferably, the material comprises tetrapodal microparticles and, optionally, fragments thereof, typically, in a smaller fraction.

Furthermore, the weight fraction of the t-ZnO microparticles and/or fragments thereof in the biocompatible structured material is about 20 to about 90 weight percent. It may be, e.g., 30-80 weight percent, 40-70 weight percent or 50-60 weight percent.

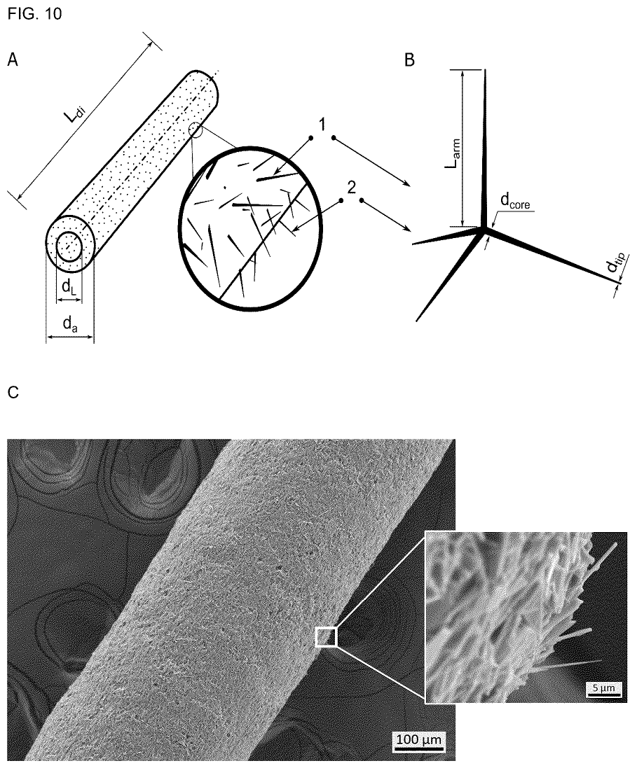

In terms of spatial distribution, the t-ZnO microparticles and/or fragments thereof of the invention are embedded in the polymerized matrix and/or are partially protruding from the matrix on the surface of the biocompatible structured material. This is illustrated in FIGS. 10, 11 and 12 and described in more detail in the Examples section. The microparticles and/or fragments typically form a substantially interconnected network structure embedded in the matrix.

In the context of the invention, “structured” means that the material is not homogenous but comprises, in addition to a matrix, the specific ZnO microparticles and/or specific micropores obtainable from removal of said ZnO microparticles. However, typically, the t-ZnO microparticles and/or fragments thereof are homogenously distributed in the matrix throughout the material. This also applies for the implantable devices of the invention.

“Biocompatible” means that the material is suitable as an implant, as further demonstrated hereon.

Preferably, throughout the invention, the polymerized matrix of the biocompatible structured material comprises an elastomer. Such elastomer may be, e.g., a poly- and/or oligo-siloxane [SiR₂O], or a mixture thereof, preferably polydimethylsiloxane (PDMS).

The polymerized matrix of the biocompatible structured material may comprise any biocompatible polymer, such as synthetic polymers, naturally-occurring polymers, or mixtures thereof. The polymerized matrix of the biocompatible structured material may also be a hydrogel.

Preferably, the elastic module of the material and the implant of the invention is at least 0.4 MPa, more preferably, at least 3 MPa, at least 10 MPa, or at least 15 MPa, e.g., 3-50 MPa, 3-21 or 15-21 MPa, e.g., as found when using poly- and/or oligo-siloxane [SiR₂O], polymers.

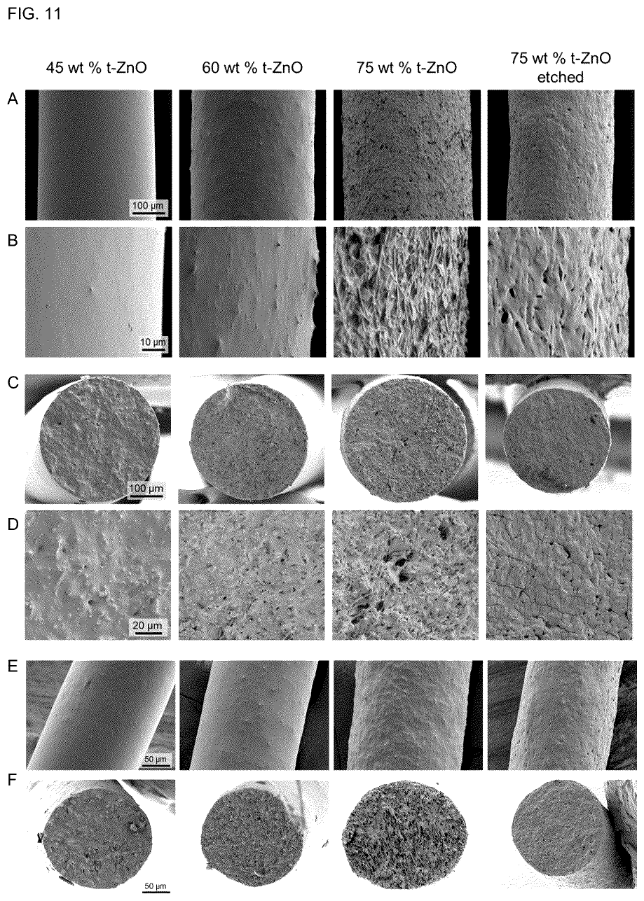

In a further embodiment, the biocompatible structured material of the invention may be subjected to acid hydrolysis (e.g., with acetic or hydrochloric acid), so as to partially (e.g., 10-90%, 20-80%, 30-70% or 40-60% or about 50%) or totally remove the substantially interconnected network structure of t-ZnO microparticles or fragments thereof from the polymerized matrix (see Examples section). The resulting biocompatible structured material (exemplified in FIG. 11) thus comprises a polymerized matrix having substantially interconnected tunnel-shaped micropores, the majority of the tunnel-shaped micropores forming a tetrahedron angle at the micropore junctions. More than 50% are a majority, but a tetrahedron angle can also be formed by more than 60%, more than 70%, more than 80% or more than 90% of junctions. In this embodiment, in other words, the matrix has interconnected tunnel-shaped micropores having a three-dimensional configuration corresponding to an interconnected hollow tetrapod network. Such tunnel-shaped micropores can have an average tunnel diameter from about 0.05 to about 5 μm and an average tunnel length from about 0.5 μm to about 100 μm. The tunnel length is determined between the micropore junctions forming a tetrahedron angle. The tunnel density of the polymerized matrix preferably ranges between about 4 and about 65 volume percent. The matrix preferably comprises an elastomer.

Microporous hydrogels comprising interconnected tunnel-shaped micropores having a three-dimensional configuration corresponding to an interconnected hollow tetrapod network suitable for reducing or eliminating motile cells from a solution or an object in contact with a solution are also disclosed in WO2016/177872 A2.

Generally, further biologically active molecules may be introduced into the biocompatible structured material by forming the biocompatible structured material in the presence of such biologically active molecules, by allowing the biologically active molecules to diffuse into the biocompatible structured material, or by otherwise introducing the biologically active molecules into the biocompatible structured material. Additionally or alternatively, the biocompatible structured material of the invention may also be coated with biologically active molecules.

Also provided herein is an implantable device comprising the biocompatible structured material of the invention. Said implantable device may be an orthopedic implant, a dental implant, a cardiovascular implant, a neurological implant, a neurovascular implant, a gastrointestinal implant, a muscular implant, or an ocular implant. It can be used to avoid, reduce or eliminate fibrotic and foreign body reaction processes in a tissue or an organ upon implantation.

More specifically, the implantable device of the invention may be a glaucoma drainage implant. Such glaucoma drainage implant may have the shape of a substantially straight, flexible, elongated body with a substantially circular or a polygonal cross-section.

In one embodiment, the glaucoma drainage implant of the invention is composed of a substantially straight, flexible, generally cylindrical body having a length preferably between about 5 and about 20 mm, and an outer diameter of preferably between about 0.1 and about 5 mm. FIGS. 10 to 12 show examples of such glaucoma drainage implants.

Further shapes for the glaucoma drainage implant of the inventions are possible, such as a substantially cuboid, flexible body having a length preferably between about 5 and about 20 mm, a width preferably between about 3 and about 10 mm, and a thickness preferably between about 0.2 and about 0.5 mm.

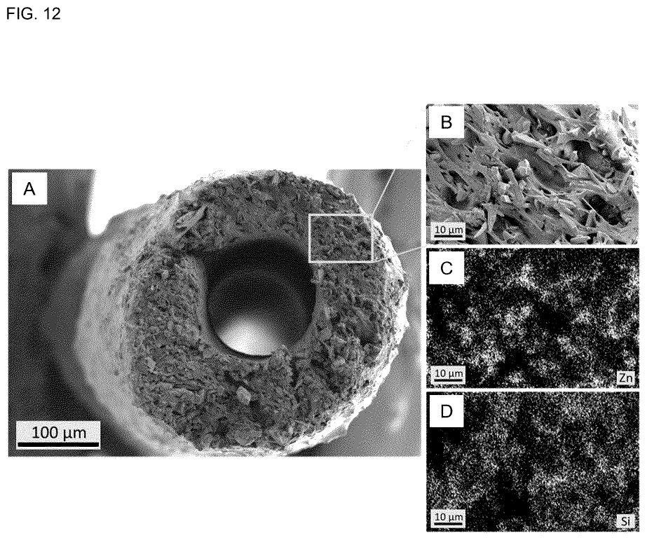

The glaucoma drainage implant of the invention may additionally contain a lumen in the body of the implant. Such a lumen is a substantially hollow channel running along the length and connecting the extremities of the body of the implant (exemplified in FIGS. 10 and 12). The position and the dimensions of such lumen can further influence the outflow of the aqueous humor beyond the effect of the outer shape of the glaucoma drainage implant itself.

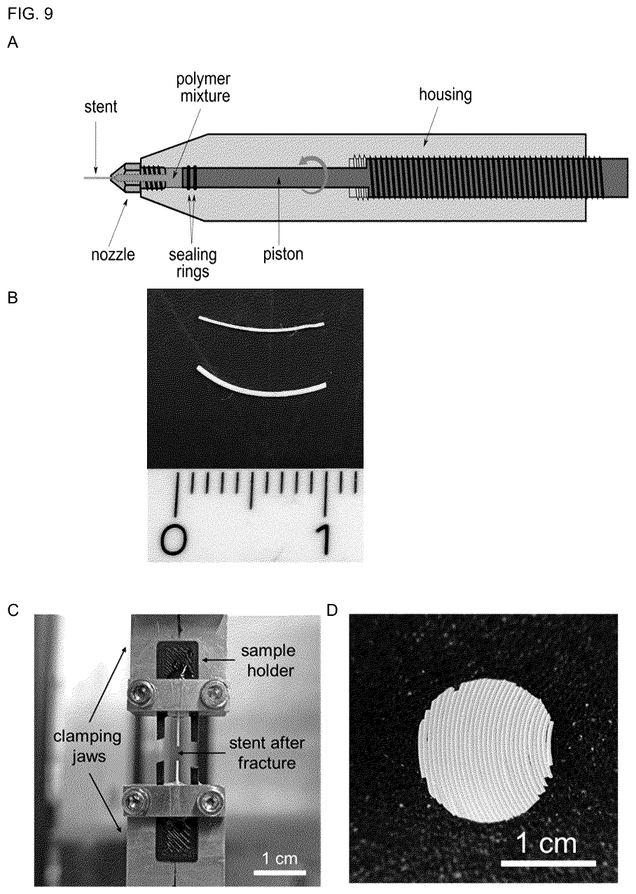

Altogether, the glaucoma drainage implants of the invention are easy and economical to produce and to sterilize by methods known from the art. Dimensionally stable, but at the same time sufficiently flexible implants according to the invention in the desired dimensions and with variable lengths can be manufactured in a single step, for example by means of extrusion processes. An example of an extrusion device for drainage implant preparation is presented in FIG. 9A. Furthermore, variable geometries and sizes can be realized using other techniques known from the art (e.g., injection molding).

The invention also provides a method for preparing an implantable device of the invention. Said method may comprise
a) providing a homogenously mixed composition comprising monomeric components of the polymerized matrix and t-ZnO and/or fragments thereof,
b) extruding said composition and letting it polymerize, and
c) cutting the polymerized material to the desired length.

In principle, the manufacturing process of a glaucoma drainage implant according to the invention may comprise the following steps:
a. A homogeneously mixed polymer composition comprising t-ZnO and/or fragments thereof is provided (depending on the proportion of t-ZnO, the mixture becomes paste-like to powder-like).
b. The polymer composition is taken into the extruder (e.g., a piston extruder).
c. The polymer composition is conveyed through a cylinder (in the case a piston extruder is used, the pressure for conveying the mixture is generated with a piston).
d. The polymer composition is forced out of a shaping nozzle.
e. The semi-finished material (filamentary in the case of a cylindrical nozzle) may be suspended between two supports and dried, e.g., in an oven.
f. After polymerization, the material is cut to the desired length.

Although the inventors have shown that, surprisingly, a glaucoma drainage implant comprising the compact biocompatible structured material of the invention leads to reduced IOP in vivo, it may be advantageous to further modify the flow properties of the implant. Thus, in addition to the steps described above, one or both of the following steps may be performed in the production of a glaucoma drainage implant according to the invention with the aim of modifying its flow properties and thus its effect on IOP:
g. A lumen may be generated by masking of a partial area during the extrusion process (e.g., using a polymer fiber or a metal wire), and by subsequently removing the masking material (e.g., by etching or mechanical extraction of the wire), so that a substantially hollow channel running along the length and connecting the extremities of the body of the implant is formed.
h. Further hollow channels in the body of the implant can be created by partially or totally etching out the t-ZnO particles with an acidic solution (e.g., acetic or hydrochloric acid). Depending on the initial t-ZnO content in the material and/or on the etching parameters, a narrow- or a wide-mesh framework structure can be obtained.

Variations of the amount of t-ZnO used in the biocompatible structured material of the invention can be used to adjust the mechanical properties of the material, such as the elastic modulus. The relatively high weight fraction of t-ZnO used in the biocompatible structured material of the invention (20-90 wt %) favors the rheological properties necessary for extrusion, as well as the sliding of the implant in the inserter device for the eye, and also facilitates the anchoring at the implantation site due to the t-ZnO particles protruding from the polymerized matrix.

The implants of the invention typically do not comprise fibers e.g., constructed from organic polymers.

Glaucoma drainage implants comprising the biocompatible structured material of present invention are particularly suited for MIGS, since they are easy to implant, they easily adapt to the shape of the implantation site and ensure long-term position stability. Upon implantation, by helping to prevent or control fibrosis around the implantation site through the reduction of cell viability, thus reducing or eliminating encapsulation processes, such glaucoma drainage implants provide controlled, long-term stable aqueous humor outflow for significant, permanent reduction of IOP, hence reducing surgical complications and extending the implants' functional timespan in situ.

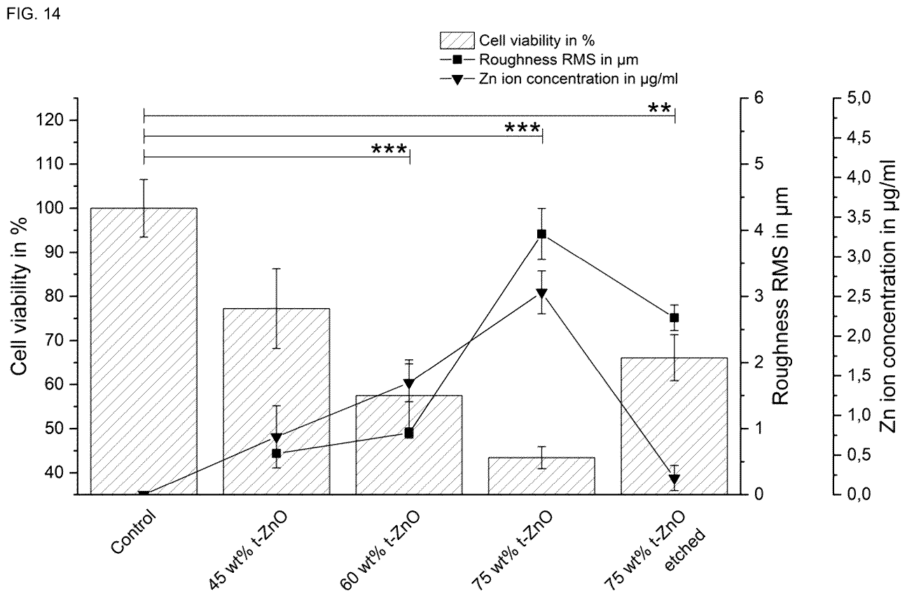

Through in vitro and in vivo studies, the inventors have demonstrated the biocompatibility (the implants were well tolerated, there was no hypotension and no clinical signs of inflammation or toxicity), the successful reduction of cell proliferation and of intraocular pressure, as well as the antifibrotic properties of the glaucoma drainage implants of the invention. The results presented in FIGS. 14 and 16 to 21 as well as in the Examples section below illustrate the significantly improved properties of such implants compared to the prior art.

Another aspect of the invention further relates to a method for reducing intraocular pressure in an eye of a mammalian subject in need thereof, the method comprising implanting one or more glaucoma drainage implants according to the invention into the eye. Such a method may be used for treating a subject having glaucoma. The method may include the steps of measuring a pre-operative IOP in an eye of the subject, implanting the glaucoma drainage implant(s) into the eye, and measuring a post-operative IOP to confirm treatment of the subject.

The invention will be further illustrated by the following examples and figures. However, these examples and figures should not be interpreted in any way as limiting the scope of the present invention.

### Examples

### Materials and Methods

1.1 Preparation of Tetrapodal ZnO Microparticles (t-ZnO)

Medical grade t-ZnO microparticles were produced at Phi-Stone AG (Kiel, Germany) in a simple and cost-effective one-step approach described elsewhere (Paulowicz et al. 2018. Zinc Oxide Nanotetrapods with Four Different Arm Morphologies for Versatile Nanosensors. Sensors and Actuators B: Chemical 262:425-435). Medical grade polydimethylsiloxane (PDMS) MED-6820 (NuSil Technology LLC, Carpinteria, USA) with a viscosity of 66 Pa*s—was used as a matrix polymer, which was provided by HumanOptics AG (Erlangen, Germany).

1.2 t-ZnO in Liquid Cultures, Absorption Spectrum

The t-ZnO was dispersed into HTF culture medium at 1 mg/mL at the start of each experiment. One reproducible standard batch of t-ZnO served as reference in all experiments and was set in relation to other samples. As t-ZnO particles precipitate quickly, the dispersion was vortexed before a serial dilution (1 to 5000 μg/mL) was prepared in culture medium. A microplate spectrophotometer (SpectraMax M4, Molecular Devices, Sunnyvale, USA) was used to evaluate the absorption spectrum of the t-ZnO dispersions in HTF-medium at 570 nm. Concentrations of 0, 0.1, 1, 10, 100 and 1000 μg/mL were tested.

### 1.3 Establishment of Human Tenon's Fibroblast (HTF) Cultures

Samples of the human Tenon's capsule were obtained from patients undergoing surgery at the University Eye Hospital, Lübeck, Germany. The study was in accordance with the tenets of the Declaration of Helsinki for the use of human tissue and informed consent was obtained from the patients after explanation of the nature and possible consequences of the study. The generation of HTF cultures was performed as described previously (Tura et al. 2007. The Rho-Kinase Inhibitor H-1152P Suppresses the Wound-Healing Activities of Human Tenon's Capsule Fibroblasts In Vitro. Invest Ophthalmol Vis Sci. 48 (5): 2152). Briefly, the tissue was dissected into 1 to 2 mm cubes and maintained in HTF-medium: DMEM/F-12 (1:1) medium supplemented with 10% heat-inactivated fetal calf serum (FCS, Invitrogen-Gibco Life Technologies, Karlsruhe, Germany), 2 mM L-glutamine, 100 U/mL penicillin, and 100 μg/mL streptomycin (Biochrom, Berlin, Germany) in a 100 mm Petri dish at 37 ° C in a humidified atmosphere with 5% CO₂. The fibroblasts migrating from these tissues were harvested after approximately 3 weeks by incubation with 0.05% trypsin and 0.02% EDTA (Invitrogen), centrifuged at 300 g for 8 minutes, and seeded in fresh culture medium in 75 cm² flasks. The cells between the third and ninth passages were used for the experiments.

1.4 Cell Viability after Incubation with t-ZnO

HTF were seeded at 5×10³ cells/well (n=6) in 96 well plates and grown to confluence for 36 hours. The t-ZnO stock solution was diluted in DMEM and added to the cell culture at different concentrations (0-15 μg/mL, 20-200 μg/mL and 1000-5000 μg/mL). HTF (passage 8) were grown initially for 36 h. The incubation period with t-ZnO was 48 h. An MTT test was performed to assess the number of viable cells (see below) and the absorbance at 570 nm was measured using a microplate reader (Tecan Group Ltd, Maennedorf, Switzerland). The standard error of the mean of three independent tests was calculated.

1.5 Ki67, α-SMA and pSMAD Immunostaining

HTFs were seeded in triplicates at a density of 3×10⁴ cells on each side of an Ibidi culture insert for live cell analysis (Ibidi, Munich, Germany) with a 500 UM separation between each side of the well, and cells were allowed to grow for 24 h. The cells were treated with different concentrations of t-ZnO (0, 2, 4, 6, 8 and 10 μg/mL). The treatment was ended after 6 hours when a complete culture medium exchange was done. 48 hours later, the cells were fixed in 2% paraformaldehyde (PFA) followed by 4% PFA for 10 minutes. Immunostaining was performed as described previously (Tura et al. 2007. The Rho-Kinase Inhibitor H-1152P Suppresses the Wound-Healing Activities of Human Tenon's Capsule Fibroblasts In Vitro. Invest Ophthalmol Vis Sci. 48 (5): 2152), using primary antibodies against Ki67 (dilution 1:300, MAB4190, Millipore, Hessen, Germany), alpha smooth muscle actin (α-SMA) (dilution 1:100, Ab7817, Abcam, Cambridge, UK) or pSMAD 2 and 3 (1 μg/mL, ab65847, Abcam, Cambridge, UK) followed by Alexa 488-conjugated anti-rabbit antibodies (diluted 1:100 in blocking buffer; Jackson Immuno-Research, Hamburg, Germany; Molecular Probes, Darmstadt, Germany, respectively). Nuclei were counterstained with DAPI (1 μg/mL in PBS) for 10 minutes. Stained HTFs were examined with an inverted microscope (Leica DMI 6000 B, Wetzlar, Germany). Photographs were captured using a DFC 290 compatible camera and the appropriate software (Leica Application Suite LAS Software, Wetzlar, Germany).

### 1.6 Quantification of Immunopositive Cells

Immunopositive cells were counted using ImageJ software. A grid was projected, then the images were initialized, and the cell counter function was activated. The mean±standard error of the mean of positive cells was calculated.

### 1.7 Wound Healing Assays

The effects of t-ZnO on primary cultures of HTF were studied using a wound healing assay with a cell insert. The HTFs were allowed to migrate freely following removal of the cell insert. Compared to the wound scratch assay, this method provides a reproducible wound of fix dimensions. In order to consider the poor solubility of t-ZnO microparticles, they were diluted in large volumes, mixed repeatedly in a vortex and multiple experiment repetitions were performed. Although the wound assay with the insert plate does not represent exactly the tissue response after injury, it allows optimal conditions to understand cell migration and proliferation. HTFs were seeded at a density of 3×10⁴ cells on each side of an Ibidi culture insert for live cell analysis (Ibidi, Munich, Germany), with a 500 UM separation between each side of the well and allowed to grow for 24 hours. The cells were treated with different concentrations of t-ZnO (0, 1, 5, 10 and 20 μg/mL). The treatment was ended after 6, 24 or 48 hours, when a complete culture medium exchange was done.

Mosaic phase contrast microphotographs were captured using the Leica DMI 6000 B microscope and Leica Application Suit LAS Software (Leica Mikrosysteme Vertrieb GmbH, Wetzlar, Germany). The use of the automated mosaic image capture allows a complete assessment of the wound gap and prevents missing or overlapping of certain areas. Images were imported to the NIH ImageJ software and the wound gap was calculated at 0, 24 and 48 hours. In order to take lost cells at the wound rim into account, the unhealed area was compared to the double wound gap area. The rate of wound healing was calculated using the following equation:

$$Rate of wound healing at 48 h = ( 1 - \frac{Unhealed wound area at 48 h}{Total wound area at 0 h \times 2} ) \times 100$$

### 1.8 Culture Supernatant Samples

HTF culture supernatant samples were collected after incubation with t-ZnO (1, 5, 10 and 20 μg/mL) for 24 or 48 hours. The control group was incubated without t-ZnO. Samples were aliquoted under sterile conditions at volumes of 50 μL, labeled and stored at −80 ° C until further use.

### 1.9 Immunoassays

To evaluate the effect of t-ZnO on the inflammatory and wound healing characteristics of HTFs, samples were examined for 5 cytokines: IL (interleukin)-1a), IL-1 B, IL-6, platelet derived growth factor (PDGF) and hepatocyte growth factor (HGF). For this, a customized fully quantitative multiplex ELISA (Q-Plex™ Human Cytokine arrays, Quansys Biosciences, Logan, UT, USA), that works as a sandwich immunoassay, was used. Each of the 5 spots within each well contains a distinct capture antibody population. The cytokine in each sample binds to their distinct capture antibody spots and subsequently to cytokine-specific, horseradish peroxidase (HRP)-bound secondary antibodies. Samples were tested using a high sensitivity protocol. Samples were diluted at 1:2 and 1:5 in Quansys human sample dilution buffer (Quansys Biosciences, Logan, UT, USA). Diluted standards and samples were added to wells containing 5-plex arrays and incubated on a plate shaker for 1 hour at room temperature. The wells were then washed 3 times with washing buffer, a detection mix was added and incubated on a plate shaker for 1 hour at room temperature. The wells were washed 3 times, then streptavidin-HRP was added for 15 min, followed by 6 washes, and a substrate for the detection of the cytokines was added. To capture the biomarker concentration in each sample, an image of the plate was taken by the Quansys Q-view imager system (Quansys Biosciences, Logan, UT, USA).

### 1.10 Scanning Electron Microscopy (SEM)

Micrographs of t-ZnO particles and stents were obtained using the SEM microscope Zeiss Ultra Plus with the Gemini column (Carl Zeiss Micros-copy GmbH, Jena, Germany) at 5 kV acceleration voltage. To prevent charging of the surface, prior to SEM analysis, the stents were sputtered with a conductive layer of gold for 90 s at 30 mA using the BAL-TEC SCD 050 Sputter Coater (Bal-Tec AG, Pfäffikon, Switzerland).

### 1.11 Statistics

Statistical analysis was performed with GraphPad Prism 6 software for Windows (California, USA). The variables distribution was evaluated with the one-sample Kolmogorov-Smirnov test. One-way ANOVA, Kruskal-Wallis, Dunn's multiple comparison, two-way ANOVA and Bonferroni post tests were used to evaluate the in vitro experiments. P-values less than 0.05 were considered as statistically significant. To estimate the half maximal inhibitory concentration (IC50), a 4-parameter logistic nonlinear regression model was used (GraphPad Prism version 6.00 for Windows, GraphPad Software, San Diego California USA). All experiments were performed in triplicate.

### 1.12 Preparation of Drainage Implants

Drainage implants were prepared by extrusion using a custom-made device illustrated in FIG. 9A. All parts except for nozzles were made of stainless steel to withstand high pressure during extrusion of the highly viscous polymer/particle mixture. As nozzles, standard MK8 brass nozzles for 3D printers with bore diameters of 400 μm and 200 μm were used, resulting in drainage implants as exemplified in FIG. 9B.

PDMS premixture was prepared by manually mixing PDMS components A and B in 1:1 ratio for at least 5 min. Next, t-ZnO microparticles were manually mixed into the PDMS premixture until a homogeneous paste- or powder-like (depending on the t-ZnO concentration) mixture was obtained. After extrusion, the drainage implants were suspended between two supports and dried in an atmospheric oven at 85 ° C overnight. After curing, the drainage implants were cut to 1.5 cm in length using a sharp blade. A minimal concentration of 45 wt % t-ZnO particles was necessary to retain the cylindrical shape of the drainage implant after extrusion and during curing. Drainage implants with 45 wt %, 60 wt %, and 75 wt % t-ZnO were produced.

Etched drainage implants were prepared by acid hydrolysis placing 75 wt % t-ZnO drainage implants in a 60% acetic acid solution for 2 days. To accelerate the etching (acid hydrolysis) process, the solution with drainage implants was placed on a heating plate at 50 °C. After etching, the drainage implants were thoroughly washed with ethanol and subsequently with water using an ultrasonic bath for 15 minutes.

The feasibility of producing a lumen in a drainage implant was demonstrated by placing a copper wire with a diameter of 100 μm inside the nozzle during the extrusion process. After curing of the drainage implant, the wire was manually pulled out.

### 1.13 Surface Characterization of the Drainage Implants

Surface morphology of the drainage implants was studied using the scanning electron microscope Zeiss Ultra Plus with the Gemini column (Carl Zeiss Microscopy GmbH, Jena, Germany) at an acceleration voltage of 4 kV. To prevent charging of the surface, the drainage implants were sputtered with a conductive layer of gold for 90 s at 30 mA using the BAL-TEC SCD 050 Sputter Coater (Bal-Tec AG, Pfäffikon, Switzerland).

Surface roughness was assessed by means of a 3D laser scanning confocal microscope VK-X (Keyence Corporation, Osaka, Japan) with a red semiconductor laser with a wavelength of 658 nm, at a magnification of 50×. The root means square (RMS) roughness was determined by averaging roughness values along 61 horizontal lines placed with an interval of 5 pixels. For each material variation/composition, three measurements on drainage implants with a nominal diameter of 400 μm were conducted.

The surface wettability was evaluated by placing a droplet of an aqueous methyl blue solution on closely aligned drainage implants and taking a photograph with a camera (Olympus TG-4, Olympus Corporation, Tokyo, Japan). The specific value of the contact angle could not be determined on individual drainage implants due to their small size and non-flat surface.

### 1.14 Tensile Testing

To access the mechanical properties of PDMS/t-ZnO drainage implants with different amounts of t-ZnO microparticles, a tensile test was conducted with drainage implants with a nominal diameter of 400 μm without a lumen. To ensure precise positioning of drainage implants and avoid slipping or premature failure at clamping jaws, the drainage implants were placed on 3D printed frames with outer dimensions of 40×8×0.5 mm (L×W×D) and a 10×5 mm (L×W) slot in the middle, similar to tabs used for tensile testing of single carbon or glass fibers. To fix the drainage implants on the frames, the drainage implants were first kept in place by using double-sided Tesa tape (Tesa SE, Norderstedt, Germany), after which one-component moisture cure silicone adhesive Elastosil E43 (Wacker Chemie AG, Munich, Germany) was applied on both ends of a drainage implant and cured overnight at room temperature. After mounting the frames into the tensile testing machine and before starting the test, both sides of a frame were carefully cut by scissors. FIG. 9 C depicts a drainage implant after breaking at maximum load.

The tensile test was performed with a BETA 5-5/6×10 tensile testing machine (Messphysik GmbH Furstenfeld, Germany) at a constant strain rate of 5 mm/min. A total of 5 drainage implants per material composition/variation was measured. Mechanical properties derived from stress-strain curves were calculated by MATLAB R2019b (The MathWorks Inc., Natick, USA). The cross-sectional area of the drainage implants for each material composition/variation was calculated using the corresponding mean diameter obtained by SEM (three drainage implants were measured for each material composition/variation, with each drainage implant measured at three different points).

1.15 In Vitro Tests with Drainage Implants

To increase the surface of contact with cells, drainage implants with a nominal diameter of 400 μm (without lumen) were placed next to each other on glass slides with a diameter of 15 mm (FIG. 9D), which were spin-coated (Specialty Coating Systems Inc., Indianapolis, USA) with a thin PDMS layer for 30 s at 2500 rpm. Three samples on glass slides were produced for each material composition/variation. The samples were then put in an atmospheric oven at 85 ° C overnight to cure PDMS and fix the drainage implants on glass slides.

For the in vitro experiments, samples were first placed in a 24-well plate, disinfected with 70% ethanol overnight and afterwards washed several times with sterile phosphate-buffered saline (PBS). 30.000 rat embryonic fibroblasts per well were seeded directly on the samples. As cell medium Dulbecco's Modified Eagle Medium (DMEM) was used (PAN-Biotech GmbH, Aidenbach, Germany), supplemented with 10% fetal bovine serum (PAN-Biotech GmbH) and 1% Penicillin/Streptomycin (Sigma-Aldrich Chemie GmbH, Taufkirchen, Germany). Negative control (cells+culture medium) and positive control (cells+culture medium+dimethyl sulfoxide (DMSO, Sigma-Aldrich Chemie GmbH, Taufkirchen, Germany) were prepared at the same time. The cells were grown to confluency for 48 hours. After that, the supernatant was removed for later Zn ion release investigations. To access the number of viable cells after incubation on surfaces with and without t-ZnO, an MTT test was conducted. 1 mL of MTT dye (3-(4,5-dimethylthiazolyl-2)-2~5-diphenyltetrazolium bromide; Sigma-Aldrich Chemie GmbH; 1 mg/mL) was added to each well and incubated for 3 hours. After washing with PBS, the samples were transferred to a new 24-well plate, DMSO was added to each well and the well plate was placed on a shaker for 5 min to solubilize formed formazan crystals. The lysates were then transferred to a 96-well plate and the absorption was measured at 570 nm using a plate reader (Epoch2, BioTek Instruments, Winooski, Vermont, USA). One-way ANOVA followed by Tukey test was performed using Origin (OriginLab Corporation, Northampton, USA).

### 1.16 Zn Ion Release Measurement

The concentration of free Zn ions (Zn²⁺) in the culture medium after the cell test was assessed by a spectrophotometric method, where Zincon monosodium salt (Sigma-Aldrich Chemie GmbH, Taufkirchen, Germany) was used as an indicator of Zn by turning the Zn ion containing solution blue at pH 9. The intensity of the blue color was measured at 620 nm using a plate reader (Tecan Group Ltd., Maennedorf, Switzerland). A set of samples with known Zn ion concentration was used to obtain a calibration curve for the determination of the Zn ion concentration in the cell culture medium samples.

### 1.17 Animal Studies

All procedures conformed to the ARVO statement of the use of animals in ophthalmic research and our institutional guidelines. The study was approved by the local committee for animal use at the University of Luebeck. All experiments were performed with female New Zealand White rabbits, 3 to 4 months old and weighing 1.5 to 2.5 kg. Animals were obtained from Charles River Laboratories (Sulzfeld, Germany) and acclimatized for at least 1 week before the experiments started. Prior to surgery, all animals were examined to exclude ocular diseases and intraocular pressure was measured with an iCare® tonometer (Icare Finland Oy, Vantaa, Finland). 12 rabbits were equally divided into 4 groups (G1-G4; Table 1). Each experimental group consisted of 3 animals. For the first set of experiments with a short follow-up of 2 weeks the animals (n=6) in two different groups (G1-G2 each n=3) differed in the implanted device. The second set of experiments was carried out with the same two types of implants with a follow-up of 6 weeks (G3-G4).

The drainage implants used in these experiments were a composite of t-ZnO microparticles (75 wt %) and PDMS. The implants used in the experimental groups differed in outer diameter (200 μm and 400 μm). A disposable inserter device with a grip, a 27-gauge slotted needle, and a deployment slide were developed to facilitate insertion of the 200 μm sized implants. The 400 μm sized implants could be easily implanted without insertion device.

TABLE 1
Animal studies, study groups

| Groups | Description |
|---|---|
| G1 | Implant with 200 μm outer diameter (n = 3) |
| G2 | Implant with 400 μm outer diameter (n = 3) |
| G3 | Implant with 200 μm outer diameter (n = 3) |
| G4 | Implant with 400 μm outer diameter (n = 3) |

### 1.18 Surgical Procedure

All rabbits (n=12) received surgery on their right eye; the left eye was used as a control. Animals were given an intramuscular injection of a ketamine-medetomidine mixture (35 mg/kg and 25 mg/kg, respectively). Additionally, all animals received topical anesthesia (conjucain eye drops). All surgeries were performed by the same surgeon and assistant using an operating microscope (Zeiss OPMI, Jena, Germany). A 90° limbus-based conjunctival peritomy was made at 10 mm from the limbus in the superotemporal quadrant and the subconjunctival space was dissected anteriorly using Westcott scissors. Diathermy was not necessary. A scleral tunnel was created with a paracentesis starting from 1.5 mm posterior to the limbus and directed to the anterior chamber. The 200 μm drainage implants were preloaded into the inserter. The preloaded inserter was then forwarded through the scleral tunnel. Once the implant was 2-3 mm into the anterior chamber, it was released from the inserter by retracting the inserter needle. The distal end was then placed under the conjunctiva (FIG. 16). The conjunctiva was closed in three cases at the opening site, because the implant appeared exposed.

### 1.19 Clinical Evaluation

Clinical examination was performed to evaluate the general appearance of the treated eyes, to assess local toxicity and ocular intolerance, and to measure the IOP. IOP was measured with the iCare® tonometer (Icare, Finland Oy, Vantaa, Finland). IOP was recorded in both eyes before surgery as the baseline and after surgery on the designated days. To exclude interindividual, cyclic, and anesthesia-related variations, IOP was compared between the experimental right eye and the left control eye. The measurements were performed in triplicate. The difference in measured IOP was expressed as the right-to-left (R/L) eye ratio. Preoperative R/L ratios ranged from 0.9 to 1.1. Success was defined by ≥20% difference in IOP, reflected as an IOP ratio of ≤0.8. Slit lamp (Keeler Itd., Berkshire, United Kingdom) follow-up was performed weekly. IOP was measured without topical anesthesia in both operated and non-operated eyes just before (day 0) in all groups and at 1, 3, 7, 10 and 14 days after surgery in groups G1-G2 and at 1, 3, 7, 10, 14, 17, 21, 24, 28, 31, 35, 38 and 42 days after surgery in groups G3-G4. Animals were examined under general anesthesia at day 14 in groups G1-G2 and at day 42 in groups G3-G4 after surgery. Statistical analysis of the IOP values was performed using SPSS 26 software (SPSS Inc., Chicago, USA). The Mann-Whitney-U-Test for independent samples was used to compare postoperative IOP ratios pairwise with the preoperative value. Levels of p≤0.05 were considered statistically significant.

### 1.20 Histological Evaluation

On postoperative day 14 (G1-G2) and 42 (G3-G4), animals were euthanized under general anesthesia with Pentobarbital (300 mg/kg body weight) and the eyes were enucleated together with the conjunctiva to preserve the bleb. The globes were immediately fixed in 10% formaldehyde for at least 24 hours. Subsequently, the eyes were examined, and a ring in the sagittal axis comprising the relevant area was excised. Tissue samples were then dehydrated and embedded in paraffin, and 5 μm serial sections were cut, rehydrated, stained with hematoxylin and eosin and the Masson technique, and cover slipped.

### Results

2.1 t-ZnO Microparticles

The arms of the t-ZnO particles exhibit a hexagonal wurtzite crystal structure oriented along the c-axis with alternating Zn²⁺ and O²⁻ stacking planes.

### 2.1.1 Scanning Electron Microscopy (SEM)

The t-ZnO microparticles have a tetrahedral geometry with 109.5u bond angles to each other. The thicknesses of the arms of the t-ZnO microparticles varied from 0.05 μm to 5 μm. Their lengths ranged from 0.5 μm to 100 μm (FIG. 1).

2.1.2 t-ZnO Absorption Spectrum

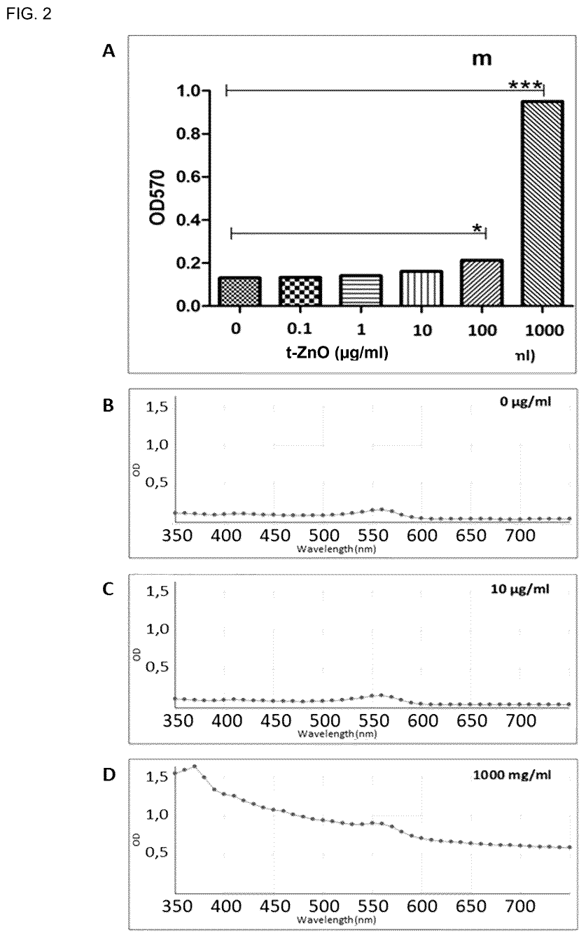

There were no significant differences in the absorption spectra of t-ZnO in HTF-medium at low concentrations (0-10 μg/mL). A significant increase in the absorption was detected at higher concentrations of 100 and 1000 μg/mL t-ZnO (p<0.05 and <0.001, one way ANOVA, Dunn's Multiple Comparison Test; FIG. 2).

2.2 t-ZnO Drainage Implants

### 2.2.1 Surface Characterization

The surface morphology of drainage implants with different amounts of t-ZnO microparticles, which were produced with a 400 μm nozzle, is shown in FIG. 11. Homogeneously distributed protruding t-ZnO microparticles on the surfaces of unetched drainage implants and distinct pores on the surface of an etched drainage implant can be observed. While single tetrapod arms are exposed from the surfaces of drainage implants containing 45 wt % and 60 wt % t-ZnO, the surface morphology of the drainage implant with 75 wt % t-ZnO is completely modified by the particles. The diameter of drainage implants (for cross-sections of drainage implants, see FIGS. 11 and 12) was found to decrease with the decreasing concentration of t-ZnO particles, which can be attributed to different viscosity during extrusion. Mean diameter values amounted to 394.5 μm±0.4 μm, 374.8 μm±0.4 μm, and 333.4 μm±0.3 μm for drainage implants containing 75 wt %, 60 wt %, and 45 wt % t-ZnO, respectively. The etched drainage implants exhibited the smallest diameter with 325.3 μm±0.3 μm due to shrinkage after removal of the t-ZnO particles. The feasibility of creating a lumen in the drainage implant by placing a metal wire inside the nozzle during extrusion is demonstrated in FIG. 12.

Given their low viscosity, liquid silicone rubbers (LSR) are typically not suitable for extrusion. In the present approach, the addition of a high amount of t-ZnO particles into liquid PDMS increases the viscosity of the PDMS/t-ZnO mixture to an extent that allows retaining the cylindrical shape in the uncured state after extrusion. Furthermore, high internal frictional forces arising from the extremely high viscosity prevent particles from “filtering”, i.e., agglomeration of the particles in the nozzle upon pressure created by a piston, and thus, prevent clogging of the nozzle and allow a homogeneous distribution of particles throughout the drainage implant. Due to flexible t-ZnO arms in the nanometer range, drainage implants with an even lower diameter of approximately 200 μm could be successfully produced with the same t-ZnO concentrations (FIGS. 11E and F). In general, an outer diameter of the drainage implant as small as possible is desired to minimize the trauma to the tissue. However, the outer dimensions of the drainage implant are determined by its inner diameter, which must ensure a sufficient flow rate, and by a certain wall thickness to maintain the structural integrity of the drainage implant. Fluid mechanical calculations showed that an optimal diameter for drainage into suprachoroidal and subconjunctival spaces is 53 μm and 40 μm, respectively. For instance, the commercially available XEN™ gel drainage implant has an inner diameter of 45 μm and an outer diameter of 150 μm (220 μm when hydrated). The techniques and methods of the present invention are suitable for fabrication of drainage implants in a similar dimension range.

### 2.2.2 Mechanical Properties

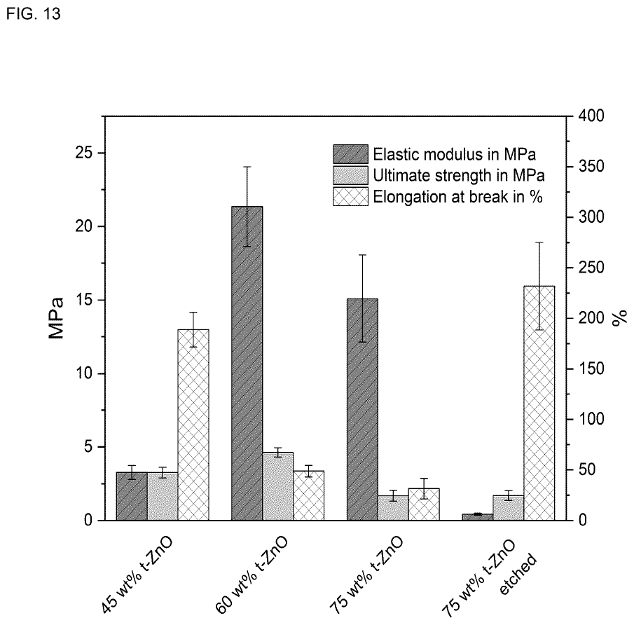

Tensile testing revealed that the incorporation of t-ZnO has a large impact on the mechanical properties of drainage implants (FIG. 13). The elastic modulus of drainage implants containing 45 wt % t-ZnO was 3.26 MPa±0.48 MPa. An increase of the t-ZnO content to 60 wt % led to an increase in the elastic modulus by a factor of 6.5 (21.34 MPa±2.72 MPa). However, a further increase in t-ZnO content to 75 wt % resulted in a decrease of elastic modulus to 15.09 MPa±2.97 MPa. The decrease of the elastic modulus can be attributed to entrapped air during extrusion, since a high amount of incorporated t-ZnO led to a nearly powder-like mixture. Less pronounced, but the same trend was observed for the ultimate strength, while the elongation at break decreased with the increasing amount of incorporated t-ZnO. Etched drainage implants exhibited the lowest elastic modulus (0.42 MPa±0.07 MPa) and the highest elongation at break (232%±43%).

A drainage implant must provide sufficient stiffness to be inserted through an injector into the target tissue, maintain the drainage implant structure, and stay in situ for a long-term period. At the same time, a drainage implant should also be flexible enough to conform to the natural curvature of an eye. It has also been shown that mechanical flexibility reduces the foreign body reaction to an implant. The flexibility of an implant can be altered by its elastic modulus, cross-sectional area, and length. In this study, drainage implants with highest amount of t-ZnO (60 wt % and 75 wt %) exhibit elastic modulus values of approximately 15-20 MPa, which is higher than that of pure PDMS, but lower than a large number of drainage implants that have been released for clinic use and have an elastic modulus in the range of 104-105 MPa. Even though the elongation at break is significantly reduced by the addition of high amounts of t-ZnO, drainage implants with 60 wt % and 75 wt % t-ZnO still show a ductile behavior exhibiting approximately 30-50% elongation before fracture.

### 2.3 In Vitro Assays

2.3.1 Cytotoxicity of the t-ZnO Microparticles

The cytotoxic potential of the t-ZnO microparticles was first investigated more closely using HTF cell cultures. To estimate the IC50, a 4 parameters logistic nonlinear regression model was used. Cell viability with the MTT test revealed an IC50 of 9.4 μg/mL (range 8,7-10,3 μg/mL; FIG. 3).

2.3.2 Ki67, α-SMA and pSMAD Immunostaining

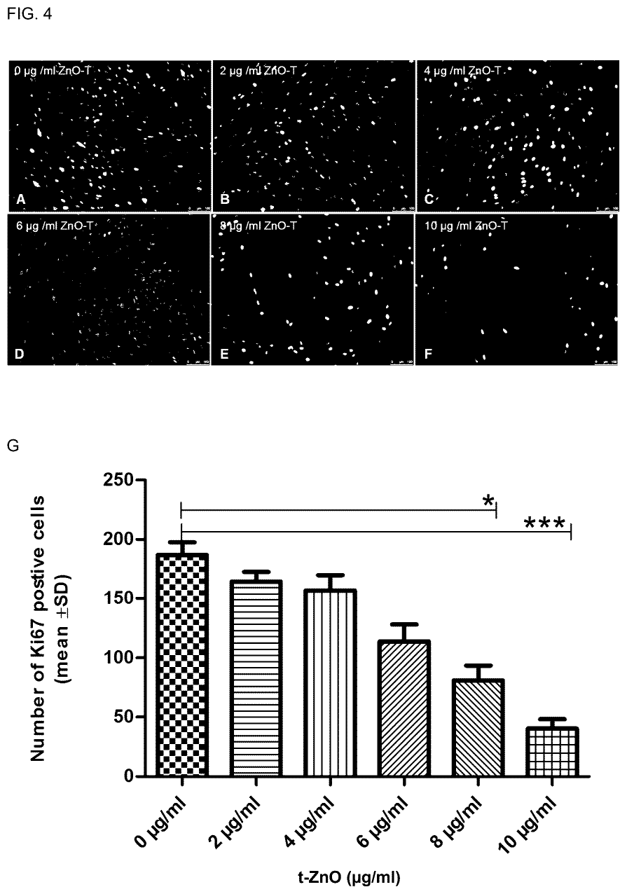

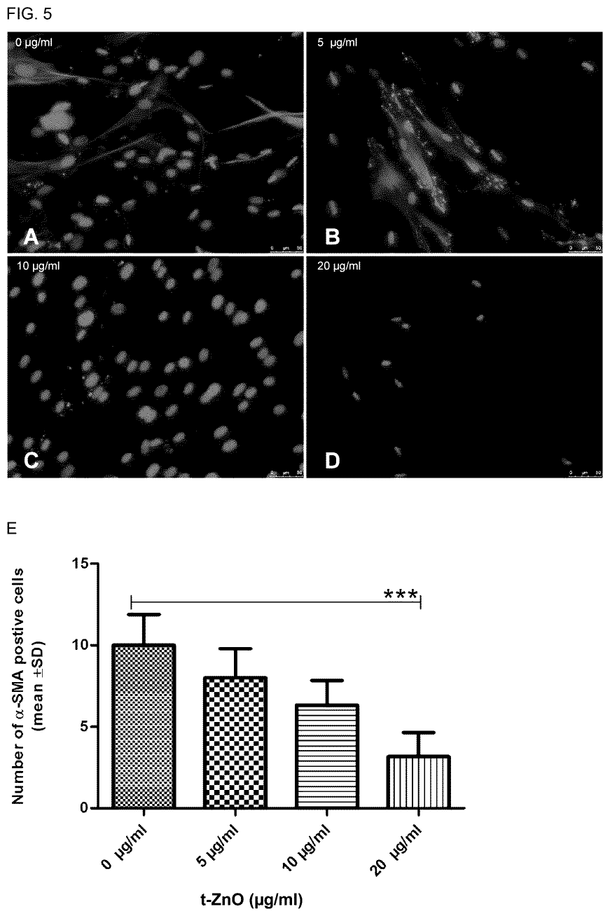

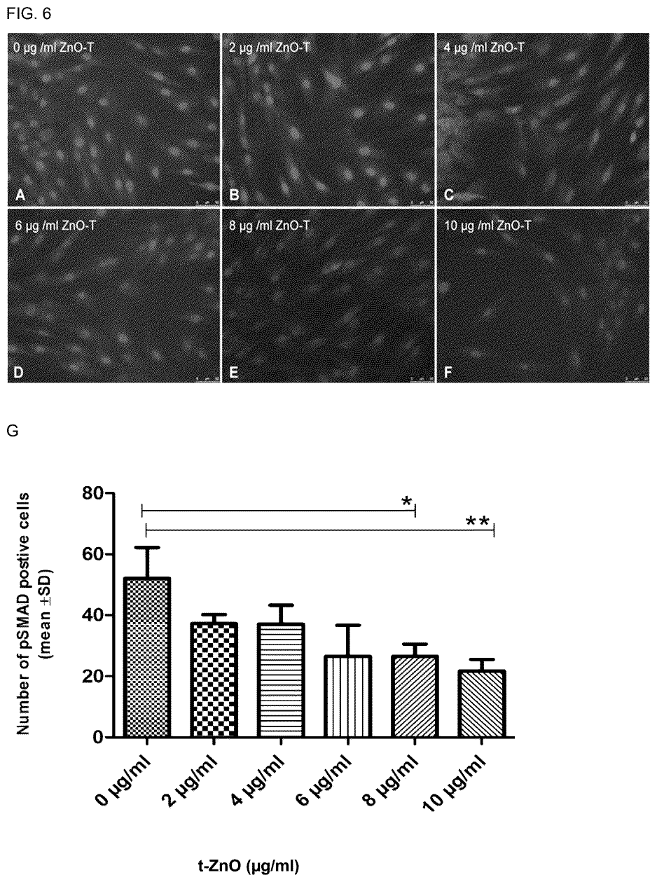

Through Ki67 staining a significant antiproliferative effect was observed after 6 h treatment with 8 and 10 μg/mL t-ZnO compared to control cells (p<0.05 and <0.001, one way ANOVA, Dunn's Multiple Comparison Test; FIG. 4). α-SMA-positive cell count as a marker for fibroblast contractility was significantly reduced after treatment with 20 μg/mL t-ZnO compared to controls (p<0.001, one-way ANOVA, Dunn's Multiple Comparison Test; Figure. 5). pSMAD-positive cells as a marker for transdifferentiation were significantly reduced after treatment with 8 and 10 μg/mL t-ZnO compared to controls (p<0.05, p<0.01, one-way ANOVA, Dunn's Multiple Comparison Test; FIG. 6).

### 2.3.4 Wound Healing Assays

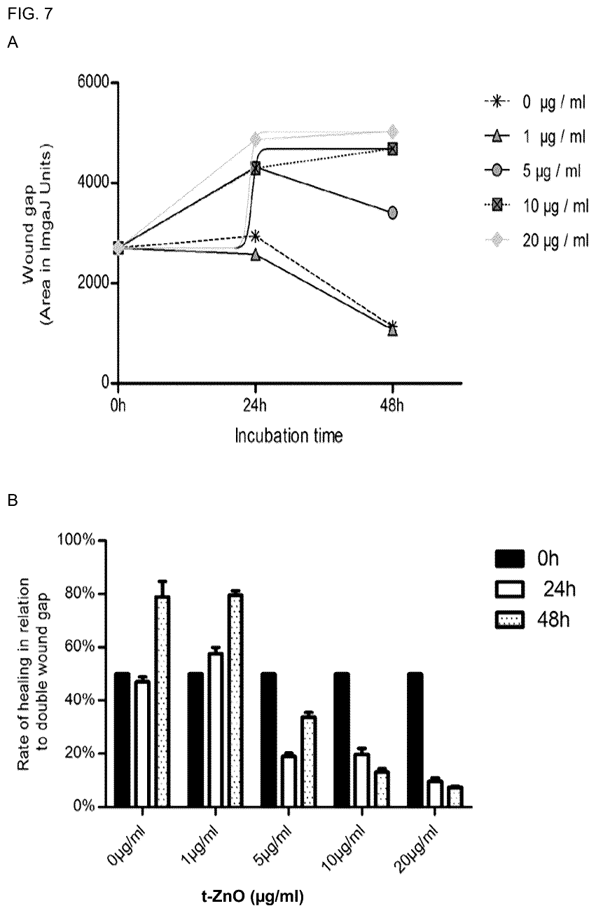

Without treatment, fibroblasts covered the scratched area within 24 hours in the performed wound healing assays. Treatment with t-ZnO microparticles resulted in a concentration and time dependent inhibition of HTF migration and proliferation. Both short (6 hours) and long-term (24 and 48 hours) treatments with t-ZnO inhibited growth and migration of the cells in a dose-dependent manner. In detail, the presented experiments show that fibroblasts tolerate treatment with concentrations of t-ZnO as high as 10 μg/mL for 6 hours without toxic effects (FIG. 3), while long-term exposure of the cells leads to significant decrease in cell count, even at the low concentration of 5 μg/mL. Application of t-ZnO for 6 hours was effective in impairing the wound healing at relatively higher doses (10 μg/mL; data not shown). With prolonged treatment, lower doses (5 μg/mL for 48 h) inhibited migration significantly, while cell toxicity became more apparent at higher doses (10 μg/mL for 48 h, FIG. 7). Migration of HTF was significantly reduced after t-ZnO treatment (10 μg/mL for 6 hrs). This migration-inhibiting effect persisted for up to 48 hours after treatment cessation and medium replacement, with no cell toxicity (data not shown). Significant reduction of cell migration compared to the control was observed after 24 and 48 hours of treatment with 5, 10 and 20 μg/mL t-ZnO (p<0.001, two-way ANOVA, Bonferroni post-test; FIG. 7). However, prolonged incubation with high t-ZnO microparticle concentrations was associated with morphological changes of HTFs that varied from loss of the spindle shape, reduction of the cytoplasm or cell death. t-ZnO microparticles were observed at the cell surface, but also embedded deeply in the cell membrane. At a t-ZnO concentration of 20 μg/mL the cells acquired a rounded form with loss of cytoplasm (not shown).

### 2.3.5 Cytokine Production: Immunoassays

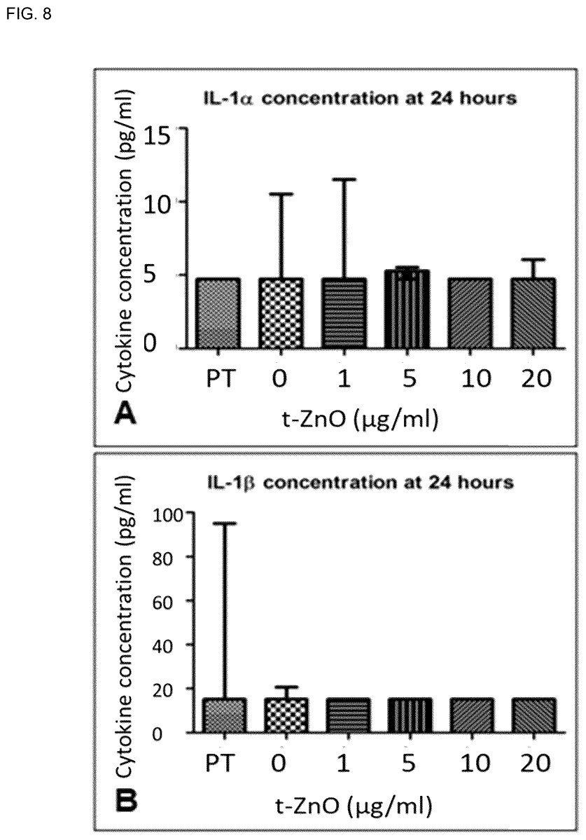

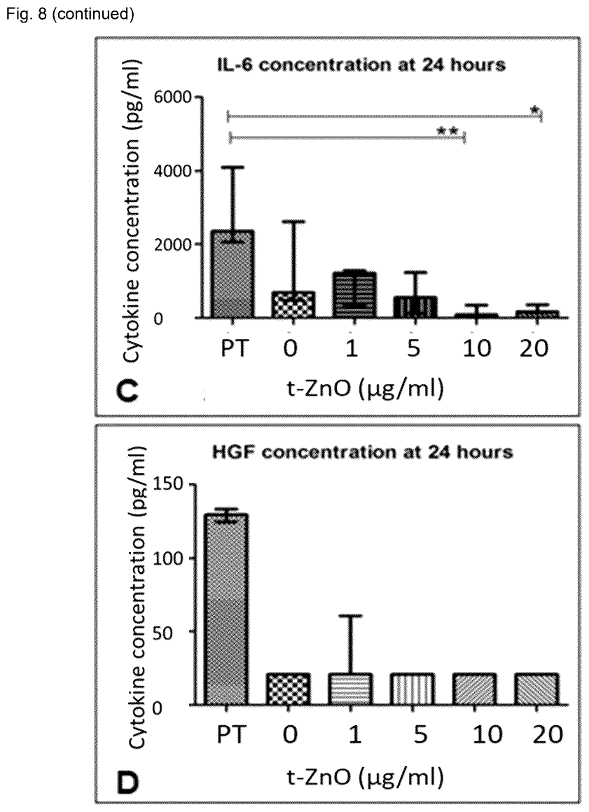

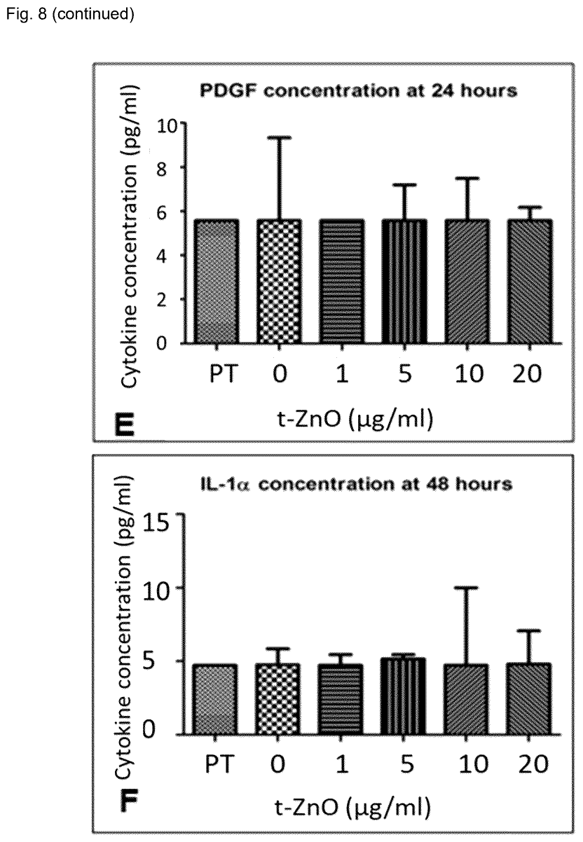

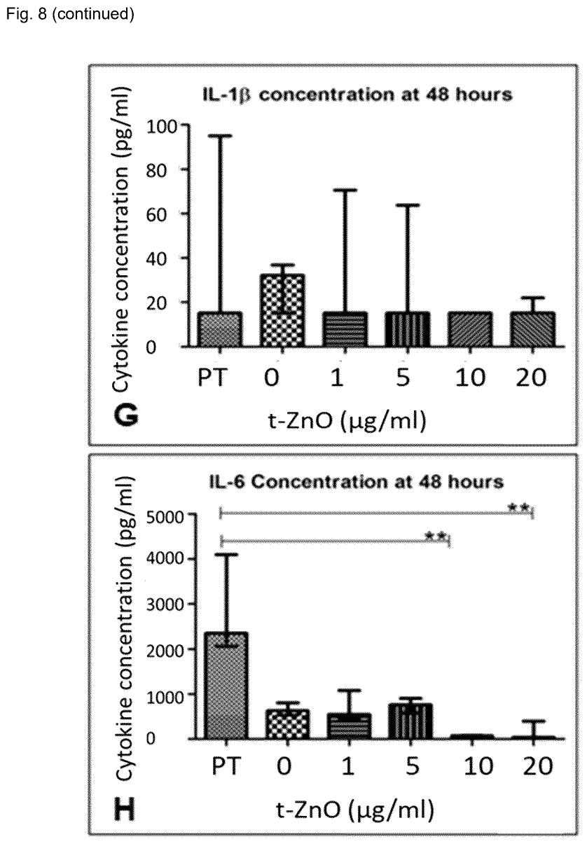

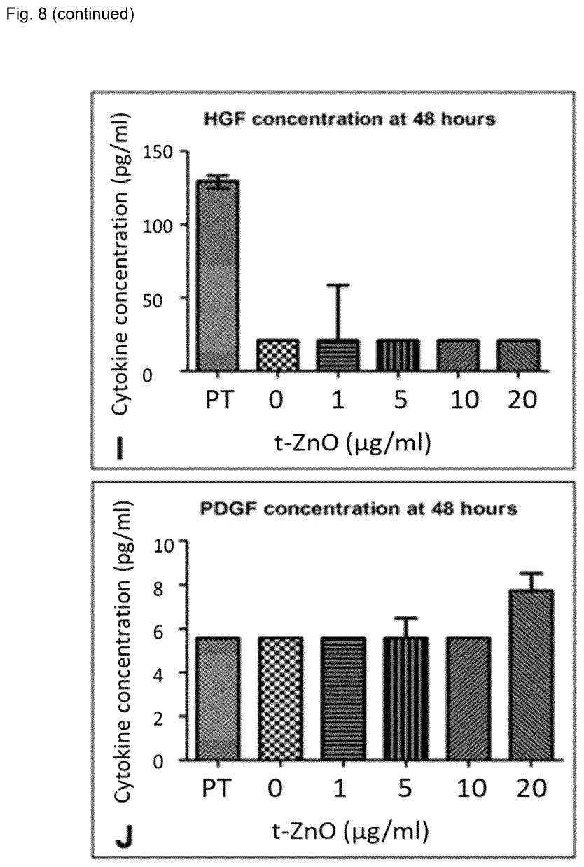

IL-6 concentration was significantly reduced in culture supernatant treated with 10 and 20 μg/mL t-ZnO for 24 and 48 hours compared to pre-treatment. (p<0.001 and <0.05, respectively, Dunn's Multiple Comparison Test; FIG. 8). There was no significant difference between the cytokine concentrations 24 and 48 hours following t-ZnO treatment (data not shown).

2.3.6 Cell Viability Assays with Rat Embryonic Fibroblasts

FIG. 14 shows that the exposure of cells to drainage implants with different amounts of t-ZnO reduced cell viability in a concentration dependent manner. The statistical analysis shows a significant reduction in cell viability on drainage implants containing 60 wt % and 75 wt % (p<0.001, indicated by three asterisks) as well as on etched drainage implants (p<0.01, indicated by two asterisks) compared to the control. Drainage implants containing 45 wt %, 60 wt %, and 75 wt % t-ZnO inhibited cell viability to 77%±9%, 57%±8%, and 43%±3%, respectively. The two latter concentrations were found to be significantly different from the control (p<0.001). Interestingly, with 66%±5%, the cell viability on etched drainage implants was also significantly lower compared to the control (p<0.01). For drainage implants containing different amounts of t-ZnO, the reduced cell viability was found to be inversely correlated with roughness and Zn ion release.

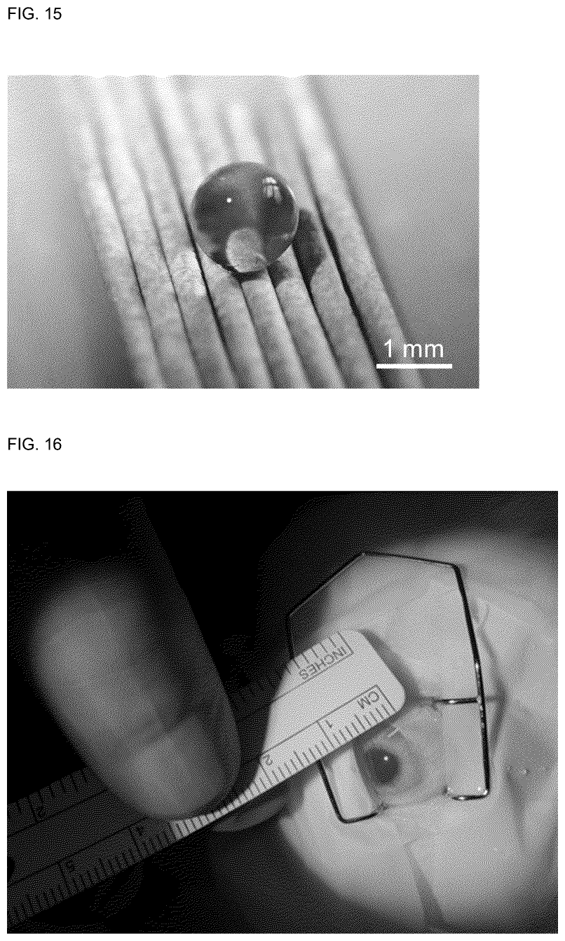

In the literature, the reported results on the fibroblast response to different surface topographies are conflicting, which could be a consequence of different substrate materials, cell types, and roughness measurement techniques and definitions used. It has to be noted that the comparability of the reported results and generalization based on average roughness values is difficult, since surfaces with different geometry and size of roughness features may yield similar roughness values. Therefore, the geometrical parameters, lateral spacing, and distribution of roughness features might be more important than average roughness values. Similarly, the initial surface wettability is enhanced by the roughness factor, i.e., the ratio between effective and projected surface area, and not by the average roughness value per se. The effect of roughness-induced superhydrophobicity, which was most pronounced on drainage implants containing 75 wt % t-ZnO, is shown in FIG. 15. An undistorted water droplet resting on the surface of drainage implants indicates a high roughness factor and possibly a wetting in the Cassie-Baxter state, where the air is trapped between the roughness features. The created roughness also facilitates the sliding of the drainage implant inside the injector, since it decreases the contact area of the soft silicone matrix with the injector walls. Furthermore, protruding t-ZnO particles may help to keep the implant in place after implantation.

In the case of t-ZnO containing drainage implants, the increasing roughness also correlates with the amount of protruding t-ZnO particles. It has been previously shown that the direct contact of t-ZnO particles with human dermal fibroblasts (NHDF) has a much higher toxic potential than in the indirect models through Zn ions (Papavlassopoulos et al. 2014. Toxicity of functional nano-micro zinc oxide tetrapods: impact of cell culture conditions, cellular age and material properties. PloS one. 9 (1): e84983). Moreover, the inventors have demonstrated the antifibrotic potential of t-ZnO by showing inhibition of HTF migration, proliferation, and transdifferentiation. It has been suggested that the toxicity occurs due to a disruption of the cell membrane by the tips of t-ZnO particles. Therefore, it is reasonable to expect that an increased amount of protruding t-ZnO particles on the drainage implant surface leads to an increase in local contacts with cells, enhancing the cell inhibiting effect.

Being an essential trace element, the second most common metal in the body (after iron) and the most abundant in the mammalian retina, ionic Zn can nonetheless exert toxic effects when present at elevated levels. Toxic effects have been observed from a concentration of ZnO particles of 10 μg/mL and higher. However, the acute toxicity of ionic Zn from ZnO is usually associated with a cellular uptake of ZnO particles, resulting in elevated intracellular Zn ion concentrations and intracellular generation of ROS, which is not the case when embedded microparticles such t-ZnO investigated in this study are used. After exposure of mouse macrophages (Ana-1) to supernatant of ZnO particles (<1 μm) suspensions and ZnCl₂, respectively the half maximal inhibitory concentration (IC50) of dissolved Zn ions at 24 h was determined as approximately 10 and 13 μg/mL (Song et al. 2010. Role of the Dissolved Zinc Ion and Reactive Oxygen Species in Cytotoxicity of ZnO Nanoparticles. Toxicology Letters 199 (3): 389-397). In the present study, the highest detected amount of dissolved Zn ions in the culture medium after 48 h was 2.55±0.27 μg/mL for the glass slide samples, which were completely covered with 75 wt % t-ZnO drainage implants (as illustrated in FIG. 9C). Therefore, the amount of Zn ions released from a single drainage implant is negligibly small. Nevertheless, at low distance, the released Zn ions could also contribute to the cell inhibiting properties at the drainage implant surface as a secondary mechanism.

### 2.4 In Vivo Assays in Rabbit Eyes

### 2.4.1 Tonometry

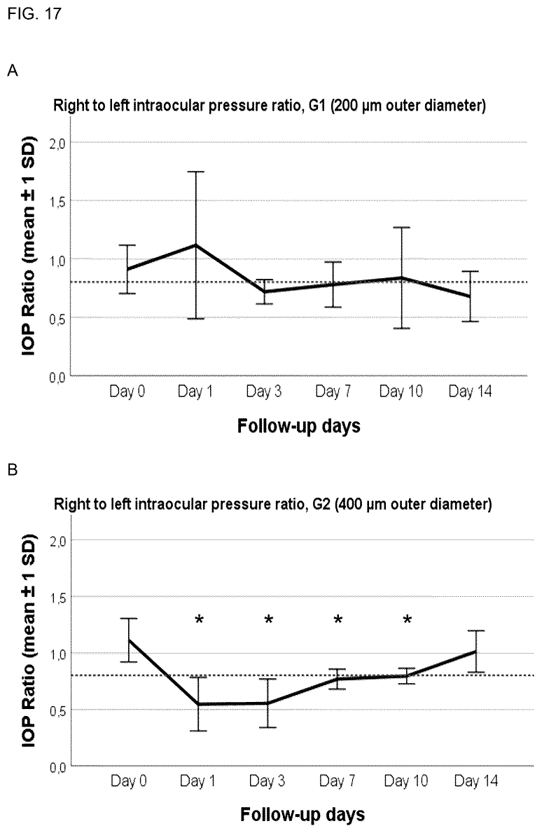

In G1, clinical success, which was defined as a 20% reduction of the IOP ratio, was observed in G1 on days 3, 10 and 14. However, no statistically significant reduction of the IOP ratio could be reached when comparing pairwise with the preoperative values (FIG. 17A). In G2, there was a significantly lower IOP ratio on days 1 (p=0.046), 3 (p=0.043), 7 (p=0.046) and 10 (p=0.043) (FIG. 17B).

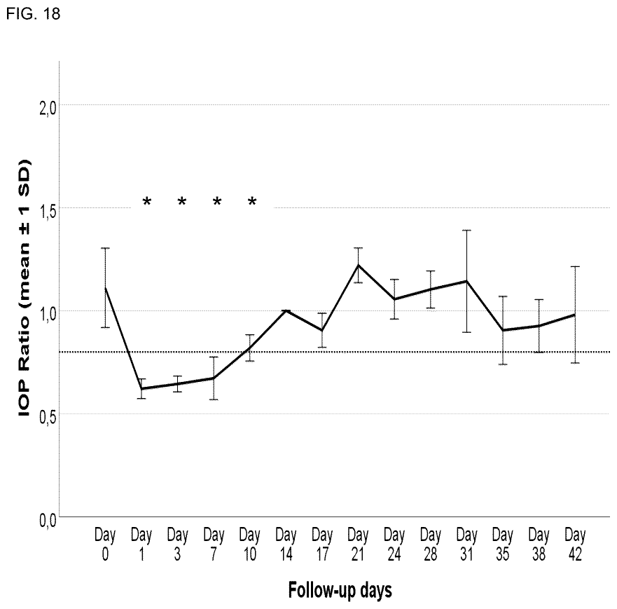

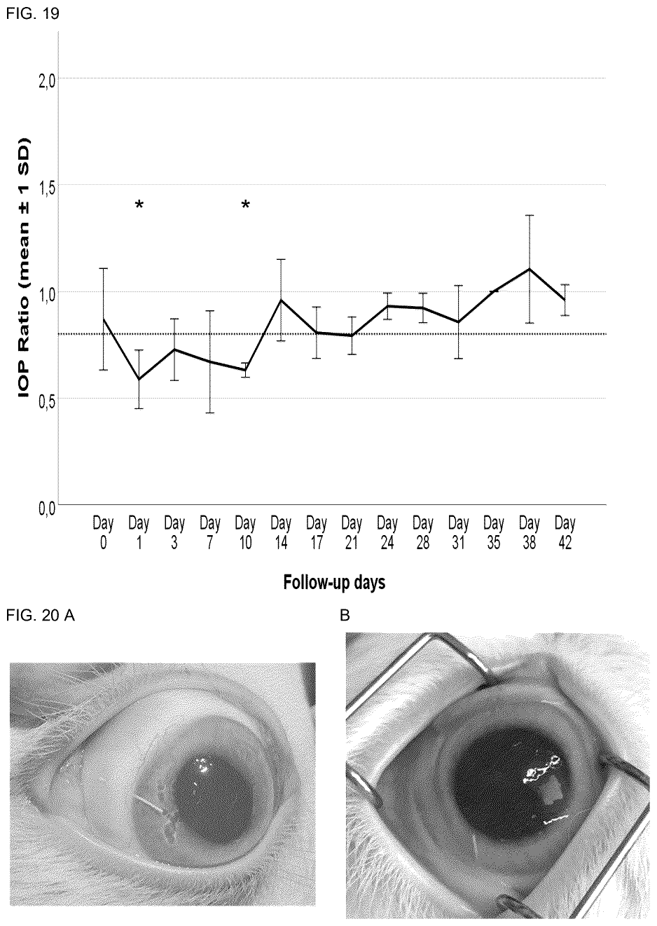

The eyes in long-term group G3 showed a significantly lower IOP ratio postoperatively up to day 10 (days 1, 7 and 10 p=0.046, day 3 p=0.043). After that, the IOP ratio was higher and even over 1 on day 21 up to day 31 (FIG. 18). The other long-term group, G4, showed a significant lower IOP ratio on day 1 (p=0.046) and day 10 (p=0.05). Furthermore, clinical success was reached on days 3, 7, 17 and 21. Afterwards, the IOP ratio increases with a maximum on day 38. During the last measurement, the IOP ratio was just below 1 (FIG. 19).

### 2.4.2 Clinical Biocompatibility

After two and six weeks, slit lamp examination of the eyes with inserted implants showed no inflammatory reactions neither in the short-nor in the long-term test. The implants were well tolerated, there was no hypotension and no clinical signs of corneal toxicity (FIG. 20). At the end of follow-up, all animals had quiet anterior chambers.

### 2.4.3 Histological Analysis

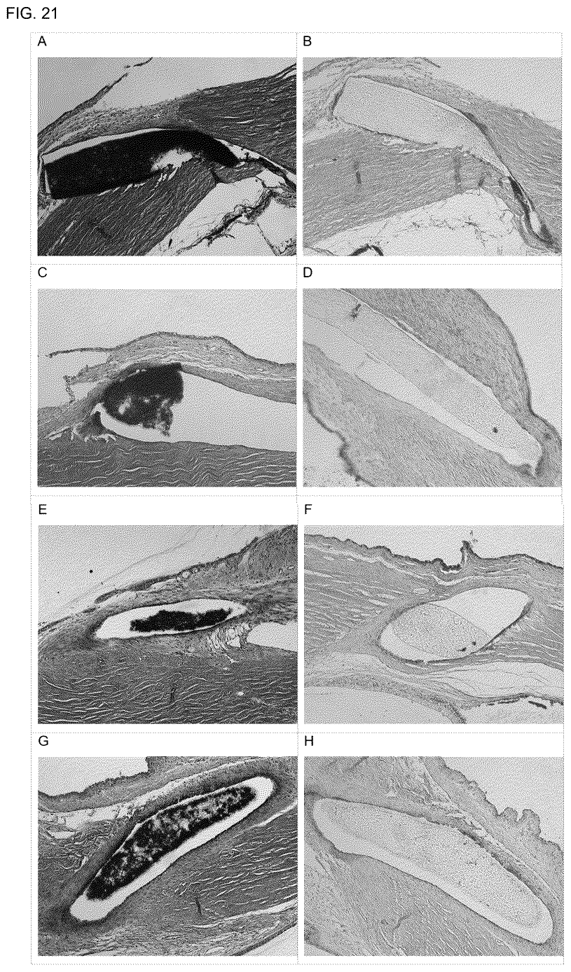

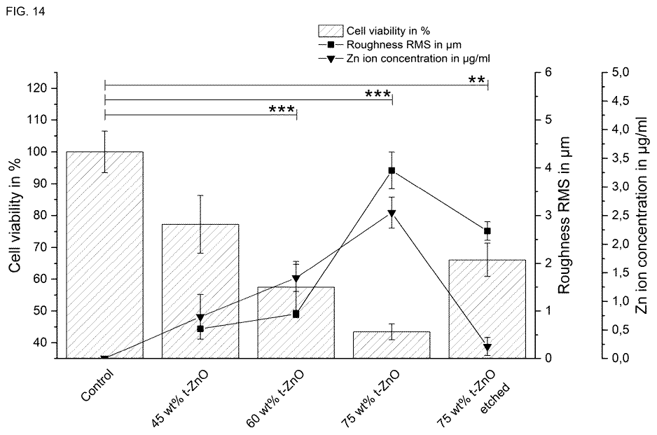

Two weeks after insertion of the drainage implants, histological analysis revealed only a very mild cell reaction: Few cells were seen at the canal wall, whereas the implant itself showed no cell colonization (FIG. 21A-D). There were no signs of inflammation or toxic changes in sensitive structures such as the corneal endothelium or the retina. In the long-term experiment, after six weeks, few cells could be observed on the implant surface. In addition, more cells were found in the wall of the canal than after two weeks, and a discrete encapsulation was observed (FIG. 21E-H). There was also no evidence of inflammation or toxic changes.

### DISCUSSION

Biocompatibility, wound healing, encapsulation and long-term success of drainage implants in glaucoma filtering surgery are still unsolved problems. Herein a new biocompatible structured material for glaucoma filtering surgery comprising a polymerized matrix and t-ZnO microparticles is described.

The inventors demonstrated that by the addition of high amounts of t-ZnO microparticles (45 wt %-75 wt % t-ZnO) into liquid PDMS, drainage implants with a diameter of 200-400 μm can be produced by extrusion techniques. Due to the high viscosity of the polymer/particle mixture, the drainage implants retain their cylindrical shape in the uncured state after extrusion. Depending on the amount of incorporated t-ZnO, drainage implants exhibit elastic modulus values from 3.3 MPa±0.5 MPa to 21.3 MPa±2.7 MPa. A lumen in a drainage implant can be created by placing a metal wire inside the nozzle during the extrusion and mechanically removing it after the curing process is completed. The addition of t-ZnO microparticles resulted in an increase in roughness (RMS) up to 3.9 μm±0.4 μm, leading to a superhydrophobic surface. The present invention provides a relatively simple and direct method for the fabrication of drainage implants with promising biological and mechanical properties, which have a great potential for application in MIGS.

In vitro experiments revealed the ability of the t-ZnO microparticles alone and in combination with a polymerized matrix to suppress cell proliferation concomitantly avoiding toxic effects.

On the one hand, in vitro experiments with rat embryonic fibroblasts revealed that cell viability was significantly reduced to 57%±8% and 43%±3% on drainage implants containing 60 wt % and 75 wt % t-ZnO, respectively. Cell inhibiting properties can be attributed to an increased amount of protruding t-ZnO particles on the implant surface, which leads to an increase in local contacts with cells and disruption of the cell membrane. As a secondary mechanism, the released Zn ions could also contribute to the cell inhibiting properties in the close vicinity of the implant surface.

On the other hand, using HTFs, the inventors showed that experimental wounds close much slower when previously incubated with the t-ZnO microparticles. t-ZnO microparticles were shown to effectively inhibit HTF proliferation, migration and transdifferentiation. Antifibrotic and anti-inflammatory properties of t-ZnO were demonstrated as suppressed expression of Ki67, α-SMA and pSMAD, as well as reduced synthesis of the cytokines IL-6 and HGF. Expression of Ki67, SMA and pSMAD was significantly downregulated in vitro following short-term treatment with t-ZnO for 6 hours, demonstrating the suppression of fibroblast proliferation, migration, and mesenchymal transformation functions. This might indicate a potential long antifibrotic effect without the need of repeated treatments.

Cell viability was only reduced at higher t-ZnO doses. Changes in cell morphology appeared to be related to the presence of t-ZnO microparticles on the cell surface. This might be the reason that surfaces displaying such t-ZnO microparticles lead to lower HTF colonization of the implant compared to the matrix material in the absence of t-ZnO. Since areas of the cell culture apart from the structured material were well covered with cells, direct contact with the t-ZnO microparticles seems to be a condition sine qua non for the suppressive effect. Therefore, it can be assumed that the mechanisms of suppression are primarily related to the morphology of the t-ZnO microparticles, representing an obstacle for the cells, which need to adhere before they can migrate and proliferate.

Thus, a brief exposure of the scleral flap and conjunctiva to t-ZnO during glaucoma surgery may be a suitable option to deliver high concentrations of t-ZnO. Extended-release systems or coated implants may be suitable to use lower concentrations of t-ZnO microparticles to suppress excessive wound healing. Prolonged treatment with high concentrations of t-ZnO (10 and 20 μg/mL) was associated with morphological changes of the fibroblasts, like the loss of their spindle shape or the reduction of their cytoplasm. As cellular uptake of the tetrapodal particles can be excluded (Papavlassopoulos et al. 2014. Toxicity of functional nano-micro zinc oxide tetrapods: impact of cell culture conditions, cellular age and material properties. PloS one. 9 (1): e84983), it is possible that the observed toxicity occurs due to a disruption of the cell membrane by the tips of the t-ZnO microparticles.

The antifibrotic effect of t-ZnO microparticles was manifested as inhibition of HTF migration, proliferation and transdifferentiation. Furthermore, the concentration of HGF in culture supernatant samples was lower compared to controls before t-ZnO application (FIG. 8). Since HGF stimulates cell proliferation, motility, morphogenesis and angiogenesis, a decrease in HGF levels may be one of the mechanisms through which t-ZnO microparticles exert their antifibrotic effect.

Lower IL-6 concentrations in culture supernatants with t-ZnO treatment indicate an anti-inflammatory effect of t-ZnO (FIG. 8). IL-6 is a pleiotropic cytokine that is involved in growth and differentiation of numerous cell types. t-ZnO treatment could suppress IL-6 production by HTF even at low concentrations of 1 μg/mL. This decrease in IL-6 may reflect the antiproliferative abilities of t-ZnO but may also be associated with its potential toxic effects at higher concentrations. The low IL-6 concentration might also reflect a milder inflammatory response following treatment with t-ZnO compared to the untreated control.

Here t-ZnO is proposed as a new antiscarring agent with the potential to contribute to an effective wound modulation following glaucoma surgery. In general, ZnO nanoparticles are among the most widely used nanomaterials in biomedicine and were recently described as selective killers for rapidly proliferating cells, whereas differentiated cells were not affected. Therefore, HTFs, which are proliferating after glaucoma filtering surgery, might also be a target for ZnO nanoparticles. However, ZnO nanoparticles might be also cytotoxic for the surrounding, non-proliferating tissue, which could cause a breakdown of the conjunctiva followed by postoperative hypotony. To overcome these potential problems, tetrapodal ZnO structures are proposed by the inventors. t-ZnO microparticles have less cytotoxic potential than spherical ZnO nanoparticles (Zarbin et al. 2010. Nanomedicine in ophthalmology: the new frontier. Am J Ophthalmol. 150 (2): 144-162.e2). Furthermore, they exhibit their cytotoxic effect through direct cell contact and only to a small extent through free zinc ions, which might be useful towards ensuring a more local antiproliferative effect and less side effects. The tetrapodal structure of the t-ZnO microparticles used in this study consists of a ZnO core in a zinc blende structure from which four ZnO arms radiate out of the wurtzite structure (FIG. 1). This relatively large, biologically active structure prevents cellular uptake and maintains the specific properties of the tetrapod tips (Papavlassopoulos et al. 2014. Toxicity of functional nano-micro zinc oxide tetrapods: impact of cell culture conditions, cellular age and material properties. PloS one. 9 (1): e84983).

Previous studies revealed an antibacterial effect of ZnO nanoparticles, for example on Staphylococcus aureus or Streptococcus agalactiae (Huang et al. 2008. Toxicological effect of ZnO nanoparticles based on bacteria. Langmuir 24 (8): 4140-4; Reddy et al. 2007. Selective toxicity of zinc oxide nanoparticles to prokaryotic and eukaryotic systems. Appl Phys Lett. 90 (213902): 2139021-3). Staphylococcus and Streptococcus species play the most important role in postoperative bleb infection and endophthalmitis. In this sense, t-ZnO microparticles may not only have a local antiproliferative effect, thus minimizing postoperative fibrosis, but may also reduce the risk of postoperative infections.

In conclusion, t-ZnO microparticles were shown to inhibit wound healing processes such as fibroblast proliferation, migration, transdifferentiation, as well as cytokine release. Thus, t-ZnO microparticles represent an innovative approach both for wound healing modulation in ocular surgery and as material for ocular implants.

The drainage implants of the invention reduced the IOP in vivo for as long as 2 weeks. A limitation of the present study is that drainage implants of the invention were not compared with other implants devoid of microparticles. However, a previous study using the same model but different implants, showed normal levels of the intraocular IOP within 1 week. This study compared a poly(styrene-b-isobutylene-b-styrene) (SIBS) drainage implant with a silicone drainage implant, which had an outer diameter of 250±10 μm and 640±15 μm, respectively. Additionally, both the SIBS drainage implant and the silicone drainage implant had an inner lumen with 65±10 μm and 300±10 μm, respectively (Acosta et al. 2006. A newly designed glaucoma drainage implant made of poly(styrene-b-isobutylene-b-styrene): biocompatibility and function in normal rabbit eyes. Arch Ophthalmol. 124 (12): 1742-1749). Nonetheless, their functional success, that is lowering of the IOP in the surgical eye, was not as long lasting as with the drainage implants of the invention.

The biocompatibility of the drainage implants of the invention was very good. Only a discrete encapsulation was found after 42 days. However, longer observation periods are necessary in order to be able to make a statement about the long-term tolerance.

Here, a drainage implant design without a lumen was used. The drainage implants currently in clinical use, such as XEN™ or Preserflo™, have a lumen. Implementation of a lumen should improve the outflow of the aqueous humor and further reduce the IOP.

In summary, both in vitro and in vivo studies with the drainage implants of the invention demonstrated more advantageous functional and biocompatibility effects than the results of comparable studies with devices that are already on the market. Nevertheless, further experiments such as long-term follow-up studies for at least several months, optimization of the implantation device and of the surgical implantation procedure are necessary before clinical testing can be started.

1-15. (canceled)

16. A biocompatible structured material comprising a polymerized matrix and tetrapodal ZnO microparticles (t-ZnO) and/or fragments thereof, wherein:
a) the t-ZnO microparticles have a core and arms having an arm length of about 0.5 μm to about 100 μm, a diameter at the core of about 0.8 μm to about 5 μm, and a diameter at the tip of about 0.05 μm to at most the diameter at the core;
b) a weight fraction of the t-ZnO microparticles and/or fragments thereof is about 20 to about 90 weight percent; and
c) the t-ZnO microparticles and/or fragments thereof are embedded in the polymerized matrix and/or are partially protruding from the polymerized matrix on a surface of the biocompatible structured material.

17. The biocompatible structured material of claim 16, wherein the polymerized matrix comprises an elastomer.

18. The biocompatible structured material of claim 17, wherein the polymerized matrix comprises poly- and/or oligo-siloxanes.

19. The biocompatible structured material of claim 17, wherein the polymerized matrix comprises polydimethylsiloxane (PDMS).

20. The biocompatible structured material of claim 16, wherein the t-ZnO microparticles and/or fragments thereof form a substantially interconnected network structure embedded in the polymerized matrix.

21. The biocompatible structured material of claim 16, wherein the t-ZnO microparticles and/or fragments thereof are homogenously distributed in the polymerized matrix throughout the biocompatible structured material.

22. A biocompatible structured material comprising a polymerized matrix having substantially interconnected tunnel-shaped micropores, wherein
a) a majority of tunnel-shaped micropores form a tetrahedron angle at micropore junctions;
b) the tunnel-shaped micropores have an average tunnel diameter from about 0.05 to about 5 μm;
c) the tunnel-shaped micropores have an average tunnel length from about 0.5 μm to about 100 μm; and
d) the polymerized matrix has a tunnel density between about 4 and about 65 volume percent.

23. The biocompatible structured material of claim 22, wherein said biocompatible structured material is obtainable by partially or totally removing, by acid hydrolysis, the t-ZnO microparticles and/or fragments thereof from the polymerized matrix of a biocompatible structured material comprising a polymerized matrix and tetrapodal ZnO microparticles (t-ZnO) and/or fragments thereof, wherein:
a) the t-ZnO microparticles have a core and arms having an arm length of about 0.5 μm to about 100 μm, a diameter at the core of about 0.8 μm to about 5 μm, and a diameter at the tip of about 0.05 μm to at most the diameter at the core;
b) a weight fraction of the t-ZnO microparticles and/or fragments thereof is about 20 to about 90 weight percent; and
c) the t-ZnO microparticles and/or fragments thereof are embedded in the polymerized matrix and/or are partially protruding from the polymerized matrix on a surface of the biocompatible structured material.

24. An implantable device comprising the biocompatible structured material of claim 16 or 22, wherein said implantable device is selected from the group consisting of an orthopedic implant, a dental implant, a cardiovascular implant, a neurological implant, a neurovascular implant, a gastrointestinal implant, a muscular implant, and an ocular implant.

25. The implantable device of claim 24, wherein the implantable device is a glaucoma drainage implant having the shape of a substantially straight, flexible, elongated body with a substantially circular or a polygonal cross-section.

26. The implantable device of claim 25, wherein the glaucoma drainage implant is composed of a substantially straight, flexible, generally cylindrical body having a length between about 5 and about 20 mm, and an outer diameter of between about 0.1 and about 5 mm.

27. The implantable device of claim 24, wherein a body of the implantable device additionally contains a lumen.

28. A method of preparing an implantable device of claim 24, comprising
a) providing a homogenously mixed composition comprising monomeric components of the polymerized matrix and t-ZnO and/or fragments thereof;
b) extruding said composition and letting it polymerize; and
c) cutting the polymerized material to the desired length.

29. The method of claim 28, further comprising creating a lumen by using a masking material selected from the group consisting of a polymer fiber and a metal wire and removing the masking material after extrusion.

30. The method of claim 28, further comprising partially or totally removing the t-ZnO particles with an acidic solution.

31. A method for reducing intraocular pressure in an eye of a mammalian subject in need thereof, the method comprising implanting one or more implantable devices according to claim 25 into the eye.
